(19) United States
(12) Patent Application Publication
(10) Pub. No.: US 2022/0049204 A1
(43) Pub. Date: Feb. 17, 2022

(54) METHODS OF MANUFACTURING CELL BASED PRODUCTS USING SMALL VOLUME PERFUSION PROCESSES

(71) Applicant: ERBI BIOSYSTEMS, INC., Stoneham, MA (US)

(72) Inventors: Harry Lee, Malden, MA (US); Kevin Lee, Arlington, MA (US)

(21) Appl. No.: 17/312,783

(22) PCT Filed: Dec. 10, 2019
(86) PCT No.: PCT/US2019/065502

§ 371 Date: Jun. 10, 2021

Related U.S. Application Data
(60) Provisional application No. 62/778,280, filed on Dec. 11, 2018.

(51) Int. Cl. C12M 1/00; C12N 5/0783; C12M 1/34

(52) U.S. Cl. CPC C12M 29/10; C12N 5/0636; C12M 41/26; C12M 41/46; C12M 41/12; C12M 41/06; C12M 41/34

(57) ABSTRACT

Methods of treating cells are disclosed. The methods include introducing a media comprising at least about 1×10⁶ cells/mL into a perfusion chamber having a volume of 50 mL or less, introducing a volume effective to treat the cells of at least one additive selected from cell culture media, a transducing agent, a pH control agent, and a cell activator into the perfusion chamber, and withdrawing cell waste and byproducts from the perfusion chamber, and harvesting the treated cells. The methods may include introducing the media comprising at least about 3×10⁶ cells/mL into the perfusion chamber. The methods may include measuring and/or controlling at least one parameter of the cells or the media selected from pH, optical density, dissolved oxygen concentration, temperature, and light scattering.

### CROSS-REFERENCE TO RELATED APPLICATIONS

This application claims priority under 35 U.S.C. § 119(e) to U.S. Provisional Patent Application No. 62/778,280 titled “Methods of Manufacturing Cell Based Products Using Small Volume Perfusion Processes” filed Dec. 11, 2018, the entire disclosure of which is herein incorporated by reference in its entirety for all purposes.

### STATEMENT REGARDING FEDERALLY SPONSORED RESEARCH

This invention was made with government support under Grant No. HHSN261201700049C awarded by the National Cancer Institute. The government has certain rights in the invention.

### FIELD OF TECHNOLOGY

Aspects and embodiments disclosed herein relate to systems and methods for treating cells. In particular, aspects and embodiments disclosed herein relate to systems and methods for treating cells for cell therapy.

### SUMMARY

In accordance with one aspect, there is provided a method of treating cells. The method may comprise introducing a media comprising at least about 3×10⁶ cells/mL into a perfusion chamber having a volume of 50 mL or less. The method may comprise perfusing the cells by introducing a volume effective to treat the cells of at least one additive selected from cell culture media, a transducing agent, a pH control agent, and a cell activator into the perfusion chamber and withdrawing cell waste and byproducts from the perfusion chamber. The method may comprise harvesting the treated cells.

In some embodiments, the media may comprise between about 5×10⁶ cells/mL and about 20×10⁶ cells/mL.

The perfusion chamber may have a volume of 20 mL or less.

The perfusion chamber may have a volume of 2.5 mL or less.

In some embodiments, the additive may comprise the pH control agent. The method may comprise controlling pH of the media within the perfusion chamber to a pH value of between about 6.8 and 7.4.

The at least one additive may be introduced at a flow rate of 5 volumes of fluid per volume of reactor per day (VVD) or less.

The at least one additive may be introduced at a flow rate of between about 1 VVD and about 3 VVD.

The method may further comprise introducing additional cells into the perfusion chamber and concentrating the cells within the perfusion chamber.

The method may comprise concentrating the cells to a concentration of at least about 5×10⁶ cells/mL.

The method may comprise concentrating the cells to a concentration of at least about 10×10⁶ cells/mL.

The method may comprise concentrating the cells to a concentration of at least about 20×10⁶ cells/mL.

In some embodiments, the harvested treated cells may have a viability of at least about 60%.

In some embodiments, the harvested treated cells may have a viability of at least about 90%.

In some embodiments, at least about 60% of the harvested cells may be effectively treated.

In some embodiments, at least about 90% of the harvested cells may be effectively treated.

In accordance with another aspect, there is provided a method of treating cells. The method may comprise introducing a media comprising at least about 0.5×10⁶ cells/mL into a perfusion chamber having a volume of 50 mL or less. The method may comprise measuring at least one parameter of the cells or the media, the at least one parameter selected from pH, optical density, dissolved oxygen concentration, temperature, and light scattering. The method may comprise determining a cell state associated with at least one of metabolic activity of the cells, average size of the cells, and density of the cells in the media, responsive to the measurement of the at least one parameter. The method may comprise introducing a volume effective to treat the cells of at least one additive selected from cell culture media, a transducing agent, a pH control agent, and a cell activator into the perfusion chamber, the volume effective of the at least one additive selected responsive to the cell state. The method may comprise harvesting the treated cells.

The media may comprise at least about 3×10⁶ cells/mL.

The perfusion chamber may have a volume of 2.5 mL or less.

The method may comprise measuring the pH and introducing a volume effective of a pH control agent to control the pH to be between about 6.8 and 7.4.

The method may comprise quantifying a volume of carbon dioxide gas introduced into the perfusion chamber to control the pH to be between about 6.8 and 7.4.

In some embodiments, the additive may comprise the transducing agent and the method further comprises introducing an effective volume of a transduction efficiency enhancing agent.

The method may comprise determining the cell state associated with metabolic activity of the cells responsive to the measurement of the at least one parameter selected from pH and optical density, and introducing the volume effective of the at least one additive selected from the transducing agent and the cell activator into the perfusion chamber, responsive to the cell state.

The method may comprise determining the cell state associated with the density of the cells in the media responsive to the measurement of the at least one parameter selected from optical density and light scattering.

In accordance with yet another aspect, there is provided a method of treating cells. The method may comprise introducing a media comprising at least about 0.5×10⁶ cells/mL into a perfusion chamber having a volume of 50 mL or less. The method may comprise perfusing the cells by introducing a first volume of at least one additive selected from cell culture media, a transducing agent, a pH control agent, and a cell activator into the perfusion chamber, after a first predetermined period of time, introducing a second volume of the at least one additive, and after a second predetermined period of time, withdrawing cell waste and byproducts from the perfusion chamber. The method may comprise harvesting the treated cells. The media may comprise at least about 3×10⁶ cells/mL.

The perfusion chamber may have a volume of 2.5 mL or less.

In some embodiments, at least one of the first and second predetermined period of time is less than about 1 hour.

In some embodiments, the first predetermined period of time may be less than about 1 minute.

In some embodiments, the first predetermined period of time may be less than about 15 seconds.

In accordance with another aspect, there is provided a method of treating cells for cell therapy. In some embodiments, the cells may be T-cells and the cell therapy point of use may be associated with chimeric antigen receptor T-cell (CAR-T) therapy.

Still other aspects, embodiments, and advantages of these exemplary aspects and embodiments, are discussed in detail below. Moreover, it is to be understood that both the foregoing information and the following detailed description are merely illustrative examples of various aspects and embodiments and are intended to provide an overview or framework for understanding the nature and character of the claimed aspects and embodiments.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a flow diagram of a method for treating cells, in accordance with one embodiment;

FIG. 2 is a schematic drawing of a perfusion chamber, in accordance with one embodiment;

FIG. 3 is a box diagram of a system for treating cells, in accordance with one embodiment;

FIG. 4 is a graph of cell density and cell viability over time, after treatment of cells in accordance with one embodiment;

FIG. 5 includes graphs of cell growth curves for comparative simultaneous perfusion cell cultures, after treatment of cells in accordance with one or more embodiments;

FIG. 6 is a graph of viable cell density and optical density over time, after treatment of cells in accordance with one embodiment;

FIG. 7 is a graph of carbon dioxide drive percentage of the cell suspension over time, after treatment of cells in accordance with one embodiment;

FIG. 8 includes graphs of pH and molecular dilution of the cell suspension over time, after treatment of cells in accordance with one embodiment;

FIG. 9 is a graph of vector copy number over time after 6 days post transduction of cells in accordance with one embodiment;

FIG. 10 is a graph of cell density and additive flow rate over time, after treatment of cells in accordance with one embodiment;

FIG. 11 is a graph of phenotype data and transduction efficiency of cells after treatment in accordance with one embodiment;

FIG. 12 is a flow diagram of a method for treating cells, in accordance with one embodiment;

FIG. 13 is a flow diagram of a method for treating cells, in accordance with one embodiment;

FIG. 14 is a flow diagram of a method for treating cells, in accordance with one embodiment;

FIG. 15 is a flow diagram of a method for treating cells, in accordance with one embodiment;

FIG. 16 is a flow diagram of a method for treating cells, in accordance with one embodiment;

FIG. 17 is a flow diagram of a method for treating cells, in accordance with one embodiment; and

FIG. 18 is a flow diagram of a method for treating cells, in accordance with one embodiment.

### DETAILED DESCRIPTION

Cell culture is a process by which cells are maintained under controlled conditions, generally in a foreign environment. Cells may be maintained, grown, activated, or transduced under controlled conditions. Conditions may vary for each process and by cell type. However, general cell culture conditions include addition of a medium that supplies essential nutrients and additives, for example, amino acids, carbohydrates, vitamins, minerals, growth factors, hormones, gases, serums, and buffers. Process specific additives may also be controlled, for example, cell activator, transducing agents, pH control agents, and others.

Cell therapy is a treatment process that generally involves administering cell products into a subject. The cell products typically include live cells. The preparations may be administered by injecting, grafting, or implanting the cell products into the subject. One exemplary cell therapy involves administering T-cells for immunotherapy treatment. T-cells may provide cell-mediated immunotherapy to the subject, for example, in the course of cancer treatment.

Cell therapy may include growing, activating, and/or transducing cells prior to administration of the cells to the subject. In certain embodiments, the cell therapy may include extracting cells and/or cell products from the subject for treatment. The extracted cells may be treated, for example, grown, activated, and/or transduced, as desired. The treated cells may be harvested and administered to the subject.

Efficiency in producing engineered cell therapies as measured by the time required to produce the therapy, quantity of reagents used, and overall effort expended may be increased by the methods disclosed herein. When performing genetic modification by transduction with viral vectors, the efficiency as measured by the number of transduced cells per virus particle, may be increased by the methods disclosed herein. The gained efficiencies allow transduction under conditions that maximize virus-cell interaction and also maximize the likelihood of genetic integration. The methods disclosed herein may increase virus-cell interactions by introducing virus through a bed of cells in flow transduction, or by increasing the density of cells per unit volume and the density of virus per unit volume. The smaller distance between particles may increase the virus-cell interaction probability. To further increase the likelihood of genetic integration, transduction may be performed on activated or dividing cells.

The systems and methods disclosed herein may be used to improve personalized cell therapy methods. Each dose of a personalized cell therapy for a subject is typically produced as a discrete manufacturing batch. Conventional manufacturing methods utilize processes and equipment designed for clinical development laboratories. Often, manual operations are performed, including cell activation, transduction, and culture media exchanges. Exemplary equipment includes static or rocking culture bags for cell expansion. Clinical research equipment for manual operations is usually open to the environment. To prevent contamination, the manufacture of a personalized cell therapy in such an environment is typically performed in an isolated biosafety cabinet. As a result, conventional methods of personalized cell therapy are generally time consuming, inefficient, and costly to the manufacturer and patient.

The systems and methods described herein may employ cell therapy processing units that may be substantially isolated from the environment. In use, the cell therapy processing units may be reversibly isolated from the environment. The substantially isolated processing units may allow multiple therapies to be produced in a bioprocessing suite while maintaining isolation.

The systems and methods disclosed herein may also be automated. Automation may reduce or eliminate manual processing steps to provide efficiencies and reduce contamination. Overall, the reduced dependence on dedicated biosafety suites and manual labor for each personalized cell therapy treatment may provide economic efficiencies to the manufacturer, reducing cost for the patient.

One cell therapy dose typically includes between 10×10⁶ cells and 250×10⁶ cells. Conventional T-cell cultures produce less than 3×10⁶ cells/mL. As a result, reactors are conventionally sized between 250 mL and 1L. The systems and methods disclosed herein may operate at high cell densities. Increasing cell density, for example, to a concentration greater than about 3×10⁶ cells/mL, may allow manufacturing in a smaller reactor, for example, having a volume of less than 100 mL. As a result, in certain embodiments, the systems and methods disclosed herein may employ reactors which produce at least 4 cell therapies per square foot of lab space, for example, at least 5, at least 6, at least 7, at least 8, at least 9, or at least 10 cell therapies per square foot. Additionally, increasing cell density may reduce the volume of liquid reagents necessary to manufacture the personalized treated cells. The high-density systems and methods disclosed herein may provide additional efficiencies by reducing sample transport distance between unit operations.

In particular embodiments, for example, in operation to produce 250×10⁶ cells in a 2 mL working volume, the systems and methods may involve processing more than 125×10⁶ cells/mL, or more than 200×10⁶ cells/mL. High intensity perfusion cultures may be employed to maintain viability of such a high-density suspension of cells, for example, by providing a substantially constant stream of fresh nutrients, while removing cell waste and byproducts.

As used herein, the subject may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, horse, goat, among others. The term “non-human animals” includes research animals, for example, mouse, rat, rabbit, dog, cat, pig, among others.

As disclosed herein, cell waste may refer to waste products produced by cells during their normal life cycle or as a result of treatment. In certain embodiments, cell waste may include dead cells and/or cell fragments. Byproducts may include secondary products produced as a result of one or more reactions in the media and unreacted products, nutrients, and additives in the media.

In some embodiments, high intensity perfusion may enable additional benefits. For example, high intensity perfusion may allow rapid removal of cell waste and byproducts. High intensity perfusion may improve transduction efficiency. For example, high intensity perfusion may allow rapid removal of viral vector. High intensity perfusion may additionally enable use of less transducing agent per cell in high-density cell environments.

Treating cells at high cell density may generally include monitoring cell metabolic activity through physiochemical sensor measurements or controller responses. In general, the signal strength of concentration dependent parameters such as pH, dissolved oxygen, or carbon dioxide may be much larger at high cell density. In some embodiments, cell metabolic activity may be monitored by monitoring and controlling pH of the media. Changes in pH controller output may be used to infer metabolic activity of the cells. Changes in pH may be measured by pH sensor or carbon dioxide or base demand of the perfusion chamber. Such monitored changes may be used in a feedback mechanism to trigger downstream or additional steps in a treatment protocol.

While embodiments described herein generally refer to gene modified cell therapies, such as chimeric antigen receptor T-cell (CAR-T) cell therapy, such an application is exemplary. It should be understood that the systems and methods disclosed may be employed for any cell treatment, including cell culture and cell therapies. For instance, systems and methods disclosed herein may be employed for treatment of stem cells (such as embryonic stem cells, mesenchymal stem cells, neural stem cells, and hematopoietic stem cells), lymphocytes (such as T-cells, B-cells, and NK-cells), blood cells (such as apheresis product and peripheral blood mononuclear cells (PBMC)), and clinical research cell lines (such as HeLa cells and MSC-1 cells). Thus, in certain embodiments, the methods may be associated with stem cell therapy. The cell therapy may involve autologous, allogeneic, or syngeneic cells.

In accordance with one aspect, there is provided a method of treating cells. The method may comprise introducing a media comprising cells to be treated into a perfusion chamber. As disclosed in the application, the perfusion chamber may be referred to as a reactor or culture chamber. The method may comprise perfusing the cells by introducing a volume effective to treat the cells of at least one additive. The at least one additive may comprise a nutrient or treatment agent. For instance, the at least one additive may comprise cell culture media, a transducing agent, a pH control agent, or a cell activator. The method may comprise withdrawing cell waste and byproducts from the perfusion chamber. The method may comprise harvesting the treated cells.

The cells may generally be introduced in a high-density suspension. For example, a concentration of at least about 3×10⁶ cells/mL may be introduced into the perfusion chamber. In some embodiments, the suspension may have a concentration of at least about 5 ×10⁶ cells/mL, at least about 10×10⁶ cells/mL, at least about 15×10⁶ cells/mL, or at least about 20×10⁶ cells/mL may be introduced into the perfusion chamber. Thus, the method may comprise introducing a media comprising between about 5×10⁶ cells/mL and about 20 ×10⁶ cells/mL into the perfusion chamber. The method may comprise introducing additional cells into the perfusion chamber. For example, cells may be introduced in multiple administrations.

The method generally includes treating very high-density cell suspensions within the perfusion chamber. Once in the perfusion chamber, the methods may comprise treating or growing the cells. During treatment, the concentration of cells may increase. In some instances, the concentration of cells may increase to be more than 5×10⁶ cells/mL, more than 20×10⁶ cells/mL, more than 50×10⁶ cells/mL, more than 100×10⁶ cells/mL, or more than 125×10⁶ cells/mL.

The method may comprise perfusing the cells with cell culture media. In particular, the method may comprise introducing a volume effective of cell culture media to maintain or grow the cells. The cell culture media may comprise one or more of minimum essential media (MEM), Dulbecco's modified eagle media (DMEM), Roswell Park Memorial Institute media (RPMI or RPMI-1640), or Iscove's Modified Dulbecco's Medium (IMDM). In certain embodiments, the cell culture media may comprise TexMACS™ T-cell culture media (distributed by Miltenyi Biotec, Bergisch Gladbach, Germany).

Additionally, the cell culture media may comprise one or more of plasma, serum, lymph, human placental cord serum, and amniotic fluid. The cell culture media may be substantially free of one or more of plasma, serum, lymph, human placental cord serum, and amniotic fluid. The cell culture media may comprise a biological buffering agent, such as phosphate buffered saline (PBS), Dulbecco's phosphate buffered saline (DPBS), Hank's Balanced Salt Solution (HBSS), and Earle's Balanced Salt Solution (EBSS). The cell culture media may be substantially free of a biological buffering agent. The cell culture media may comprise an acid or a base. The cell culture media may comprise essential nutrients for cell viability, such as, amino acids, carbohydrates, vitamins, minerals, growth factors, hormones, tissue extracts, and dissolved gases. In certain embodiments, the cell culture media may comprise a cytokine signaling molecule. For example, the cell culture media may comprise IL-2, IL-7, IL-15, or combinations thereof, for treatment of T-cells. The cell culture media may comprise Laminin-111 for treatment of embryonic stem cells.

The method may comprise inoculating the perfusion chamber with the media comprising the cells by introducing the suspension into the perfusion chamber. The method may comprise mixing or agitating the cell suspension to perfuse or maintain the cells. In some embodiments, the mixing or agitating may be performed intermittently. For example, the method may comprise mixing or agitating the suspension in 1-10 cycles, for example, 3-5 cycles. The method may comprise delaying each cycle by up to about 5 seconds, up to about 10 seconds, up to about 15 seconds, or up to about 30 seconds. The method may comprise mixing or agitating the suspension at a frequency of between about 1.5 Hz and about 5 Hz.

The method may comprise perfusing the cells with an additive comprising a cell activator. The additive may comprise a cell activator suitable for the cell type to be treated. For instance, the cell activator may comprise magnetic beads, mitogen-based activators, soluble and/or plate or particle-bound antibodies (for example, human CD2, CD335, CD3, and/or CD28 antibodies), and antigen presenting cells (APC). In exemplary embodiments, the cell activator may comprise magnetic Gibco Dynabeads™ (distributed by Thermo Fisher Scientific, Waltham, Mass.), Anti-Biotin MACSiBead™ Particles loaded with biotinylated antibodies (distributed by Miltenyi Biotec, Bergisch Gladbach, Germany), or TransAct™ colloidal polymeric nanomatrix structure conjugated to humanized antibody agonists (distributed by Miltenyi Biotec, Bergisch Gladbach, Germany), for treating human T-cells.

The method may comprise introducing the cell activator until the media comprises at least about 10×10⁶ activated cells/mL. In other embodiments, the method may comprise introducing the additive comprising the cell activator until the media comprises at least about 25×10⁶ activated cells/mL, at least about 50×10⁶ activated cells/mL, at least about 75×10⁶ activated cells/mL, at least about 100×10⁶ activated cells/mL, at least about 125×10⁶ activated cells/mL, at least about 150×10⁶ activated cells/mL, 175×10⁶ activated cells/mL, or 200×10⁶ activated cells/mL. In general, the method may comprise introducing the cell activator until the media comprises a target amount of activated the cells. The target amount of activated cells may be substantially the same as the target amount of treated cells.

The method may comprise introducing at least two boluses of the cell activator. As used herein, a bolus may refer to a discrete amount of additive to be introduced in one administration, or within a preselected time period. The preselected time period may be, for example, within 1-10 minutes or within 1-5 minutes. In general, a bolus administration may be a continuous administration of the discrete amount. Thus, the method may comprise introducing a first dose of the cell activator, after a period of time introducing a second dose of the cell activator. The period of time may be greater than about 5 minutes, greater than about 10 minutes, greater than about 15 minutes, greater than about 20 minutes, or greater than about 30 minutes, depending on the cell type, cell density, and protocol.

Conventionally, after an activation cycle, treatment protocols may recommend splitting cells into low-density cultures to replenish spent media and then re-activating the cells with a low-density expansion protocol. The methods disclosed herein may comprise re-activating and/or expanding cells at the high density. Such methods may reduce handling and processing time. The method may comprise concentrating the cell activator within the perfusion chamber. For instance, the method may comprise concentrating the cell activator by a factor of 2, 5, 10, 25, or 50. In certain embodiments, cell activator can be introduced into the perfusion chamber and concentrated with a retaining filter to deliver the target concentration of cell activator to the high-density cell culture. In other embodiments, the cell activator may be introduced at a high flow rate perfusion to deliver the target concentration of cell activator.

The method may comprise perfusing the cells with an additive comprising a transducing agent. Transduction may generally refer to the process by which DNA is introduced into a cell. Typically, DNA is introduced through transduction with a virus, viral vector, or viral particle. A transducing agent having a plasmid encoding the target DNA may be introduced in an amount effective to infect the cells leading to expression of the target DNA. In some embodiments, the transducing agent may insert the target DNA into the cell's genome. The transducing agent may comprise lentivirus, retrovirus, adenovirus, adeno-associated virus (AAV), transposon, mRNA electroporation, and hybrids thereof coding the target DNA. In general, lentivirus and retrovirus may integrate the target DNA into the cell genome and replicate during cell division.

The effective amount of the transducing agent may be at least 50% less than a concentration effective to transduce cells at a cell density lower than about 3×10⁶ cells/mL.

The method may comprise introducing the transducing agent until at least about 60% of the activated cells are effectively transduced. In other embodiments, the method may comprise introducing the transducing agent until at least about 70%, about 75%, about 80%, about 85%, about 90%, or about 95% of the viable cells are effectively transduced. For instance, the method may comprise introducing a volume effective of the transducing agent to effectively transduce at least about 60%, about 65%, about 70%, about 75%, about 80%, about 85%, about 90%, or about 95% of the viable cells.

The method may comprise introducing at least two boluses of the transducing agent. As previously described, a bolus may refer to a discrete amount of additive to be introduced in one administration, or within a preselected time period. The preselected time period may be, for example, within 1-10 minutes or within 1-5 minutes. In general, a bolus administration may be a continuous administration of the discrete amount. Thus, the method may comprise introducing a first dose of the transducing agent, after a period of time introducing a second dose of the transducing agent. The period of time may be greater than about 5 minutes, greater than about 10 minutes, greater than about 15 minutes, greater than about 20 minutes, or greater than about 30 minutes, depending on the cell type, cell density, and protocol.

The method may further comprise introducing an effective volume of a transduction efficiency enhancing agent. The transduction efficiency enhancing agent may comprise, for example, a cell and virus co-location agent. The co-locating agent may comprise a reagent with multiple binding domains for virus and cells. One such exemplary co-locating agent is RetroNectin® reagent (distributed by Takara Bio Inc., Kusatsu, Shiga Prefecture, Japan). The transduction efficiency enhancing agent may comprise, for example, a non-ionic surfactant. One exemplary non-ionic surfactant is Synperonic® F108 surfactant (distributed by MilliporeSigma, St. Louis, Mo. USA). The transduction efficiency enhancing agent may comprise, for example, a cationic polymer. The cationic polymer may enhance transduction efficiency by neutralizing the charge repulsion between agents and cells. One exemplary cationic polymer is hexadimethrine bromide (distributed under trade name “polybrene” by MilliporeSigma, St. Louis, Mo. USA).

The method may generally comprise performing various operations in sequence. In some embodiments, the method may comprise one or more of introducing the cells in media; inoculating the cells in the perfusion chamber; mixing or agitating the cell culture; performing liquid exchange to replace media; introducing an additive, for example, nutrients, viral vector, or activation reagent, optionally through precise fluid injection; cell-free removal of liquid, optionally through a cell retention filter; viral vector-free removal of liquid, optionally through a virus retention filter; removing and harvesting of cell samples, optionally less than 5-10% of the working volume; and measuring and controlling pH, dissolved oxygen, optical density, and/or temperature.

In certain embodiments, the method may comprise continuously perfusing the cells with media, optionally including one or more additive. Continuous perfusion may generally comprise introducing the media in short pulses, approximating uninterrupted perfusion. For instance, continuous perfusion may comprise introducing a first volume of media and, after a short predetermined period of time, introducing a second volume of media. Continuous perfusion may comprise removing media, optionally retaining cells after some number of pulses have been added. The predetermined period of time may be less than about 1 hour, less than about 5 minutes, less than about 1 minute, less than about 30 seconds, less than about 20 seconds, less than about 15 seconds, or less than about 10 seconds. The volume of media for each administration may comprise between 0.1% and 25% of the total volume of media for perfusion. After adding pulses of fluid, the method may comprise withdrawing the cell waste and byproducts from the perfusion chamber. For example, the method may comprise withdrawing the cell waste and byproducts after more than 5 pulses, or more than 10 pulses, or more than 50 pulses, or more than 100 pulses of fluid.

In certain embodiments, for example, in cell therapy applications, the method may comprise sequentially perfusing the cells with more than one additive. For instance, the method may comprise continuously perfusing the cells with a volume effective to culture the cells of the cell culture media, continuously perfusing the cells with a volume effective to activate the cells of the cell activator, and continuously perfusing the cells with a volume effective to transduce the cells of the transducing agent.

The cells may be continuously perfused with cell culture media for a period of time sufficient to nurture and/or inoculate the cells within the perfusion chamber. The cells may be continuously perfused with cell activator for a period of time sufficient to activate and/or expand a target amount of the cells, for example, at least about 60%, about 70%, or about 90% of the viable cells. The cells may be continuously perfused with cell transducing agent for a period of time sufficient to effectively transduce a target amount of the cells, for example, at least about 60%, about 70%, or about 90% of the viable cells.

In some embodiments, the cells may be mixed or agitated during any one or more of cell culture, activation, expansion, and transduction. After any one or more of cell culture, activation, expansion, and transduction, or as necessary, the method may comprise withdrawing the cell waste and byproducts from the perfusion chamber. In some embodiments, the method may comprise withdrawing cell waste and byproducts from the perfusion chamber concurrently or consecutively with any of the steps described herein. In general, the cells may remain in the perfusion chamber while cell waste and byproducts are withdrawn.

In some embodiments, each cycle may independently be performed for a predetermined period of time. Thus, each of the cell culture, activation, expansion, and transduction may independently be a pre-selected period of time. In other embodiments, each cycle may be performed responsive to a measurement of at least one parameter, as described in more detail below. In yet other embodiments, at least one of cell culture, activation, expansion, and transduction may be performed for a predetermined period of time based on historical data of the measured parameters.

In some embodiments, the cells may be harvested from the perfusion chamber less than 7 days after the transducing agent is introduced. The cells may be harvested from the perfusion chamber less than 6 days, less than 5 days, less than 4 days, less than 3 days, less than 2 days, or less than 1 day after the transducing agent is introduced.

The cell treatment from introduction of the cells in media into the perfusion chamber through harvesting the cells may be performed in less than about 3 weeks. In some embodiments, the cell treatment may be performed in less than about 2 weeks, in less than about 1 week, in less than about 5 days, in less than about 3 days, or in less than about 1 day. The period of time to complete the cell therapy may generally depend on the density of cells introduced and whether the cells are introduced into the perfusion chamber in an activated state. For instance, in certain embodiments, between about 3×10⁶ cells/mL and about 5×10⁶ cells/mL may be introduced into the perfusion chamber prior to cell activation. In such embodiments, the cell treatment may be performed in about 1-3 weeks. In other embodiments, between about 10×10⁶ cells/mL and about 30×10⁶ cells/mL may be introduced into the perfusion chamber with a cell activator. In such embodiments, the cell treatment may be performed in about 3 days-1 week.

Any of the reagents may be introduced at a substantially constant flow rate. In other embodiments, the reagents may be introduced at a variable flow rate. For instance, flow rate of a given additive may increase in subsequent cycles, with increasing cell density. Flow rate of the reagent may be correlated with the effective amount of any given reagent, as generally the net amount of the reagent introduced may be increased or decreased for a given period of time by increasing or decreasing flow rate of perfusion.

Flow rate of the reagent being perfused may be reduced by the methods disclosed herein, as compared to conventional perfusion methods (for example, methods of perfusing cells at a density lower than 3×10⁶ cells/mL). In some embodiments, reducing flow rate of the reagent may increase contact time between the cells and the at least one additive being administered. In high cell density suspensions, increased contact time may improve viability and rate of treatment of the cells. in some embodiments, the at least one additive may be introduced at a flow rate of 10 volumes of fluid per volume of reactor per day (VVD) or less. For instance, the at least one additive may be introduced at a flow rate of between about 1 VVD and about 5 VVD, or between about 1 VVD and about 3 VVD.

In some embodiments, fluids may be replaced in the perfusion chamber in stepwise cycles. For example, a predetermined amount of fluid, optionally cell-free fluid, may be withdrawn from the perfusion chamber before introducing a substantially equivalent amount of fluid with the at least one additive. The fluids may be introduced and/or withdrawn by a precise fluid injection. The precise fluid injection may comprise, for example, administering or withdrawing fluid with a syringe. Other embodiments are discussed in more detail below. In some embodiments the fluid may be replaced in discrete amounts of between about 10 μL and about 500 μL. The total amount to be replaced may be selected based on a desired concentration of one or more additive in the replacement fluid. If the desired concentration is great, the method may comprise performing more than one discrete fluid replacement step to achieve the desired concentration. The fluid may be replaced in discrete amounts of between about 1% and about 25% of the total volume within the perfusion chamber. For example, the fluid may be replaced in discrete amounts of between about 1% and about 10% of the total volume within the perfusion chamber.

The harvested cells may have a viability of at least about 60%. In particular, the conditions in the perfusion chamber may be controlled such that the harvested cells have a viability of at least about 60% at the time of harvesting. The harvested cells may have a viability of at least about 70%, at least about 75%, at least about 80%, at least about 85%, at least about 90%, at least about 95%, or at least about 99%. Conditions such as cell density, temperature, and additive concentration may be controlled to provide the desired cell viability of the harvested cells.

The methods may comprise controlling pH of the media to monitor and/or control cell metabolic state. In such embodiments, the methods may comprise introducing an effective amount of an additive comprising a pH control agent. The method may comprise controlling pH of the media within the perfusion chamber to a pH value of between about 6.0 and 8.5, for example, between about 6.8 and about 7.4. The methods may comprise controlling pH to a substantially physiological pH value. In some embodiments, the pH control agent may be a base. In some embodiments, the pH control agent may be an acid. The pH control agent may comprise, for example, sodium hydroxide, sodium carbonate, sodium bicarbonate, ammonia, potassium hydroxide, carbon dioxide, hydrochloric acid, or phosphoric acid.

While not wishing to be bound by theory, it is believed that cell activation for a high-density culture (for example, more than 2×10⁶ cells/mL) causes a large change in media pH due to the increase in cellular metabolism. Maintaining the pH at acceptable levels (for example, between approximately 6.9 and 7.3 for T-cells) may be essential for cell growth and viability during unit operations where high cell density is advantageous, such as cell transduction, and cell expansion. Perfusion flow may counteract the metabolic byproducts (typically acidic in nature but may be basic) generated by the cells. For instance, perfusion flow may control or reduce the change or decrease in pH compared to batch cultures.

However, excessively high perfusion rates may exceed the flow rate supported by a perfusion filter, or excessively dilute the culture media of paracrine factors such as cytokines or viral vector. In some embodiments, a pH control agent may be added to prevent the change in pH of the culture media. The pH control agent may permit control of pH with a lower perfusion rate. By combining perfusion with active pH control during cell activation, transduction, and/or expansion, perfusion rate may be controlled independently from pH control. Flow rates may be reduced to less than 20 volumes of fluid per volume of reactor per day (VVD), for example, less than 10 VVD, less than 5 VVD, less than 2 VVD, less than 0.5 VVD, or lower.

At least about 60% of the harvested cells may be effectively treated. In particular, the conditions in the perfusion chamber may be controlled such that at least a target percentage of the cells are effectively treated at the time of harvesting. In some embodiments, at least about 70%, at least about 75%, at least about 80%, at least about 85%, at least about 90%, at least about 95%, or at least about 99% of the harvested cells may be effectively treated.

Conditions such as cell density, temperature, and additive concentration may be controlled to provide the target percentage of effectively treated cells.

The method may comprise introducing a volume effective of a cell culture media comprising at least one nutrient or dissolved gas to maintain viability of the cells to at least about 60%. For example, the method may comprise introducing a volume effective of the media comprising at least one nutrient or dissolved gas to maintain viability of the cells to at least about 70%, at least about 75%, at least about 80%, at least about 85%, at least about 90%, at least about 95%, or at least about 99%. The volume effective to maintain a target percentage viability may be at least partially dependent on factors such as cell density, temperature, and state of the cells.

The methods disclosed herein may be used for treating cells for cell therapy. The methods may comprise delivering the treated cells to a cell therapy point of use. In exemplary embodiments, the cell therapy point of use may be associated with CAR-T therapy. Thus, the cells may comprise lymphocytes. For instance, the cells may comprise T-cells. The methods may comprise activating the cells with one of magnetic Gibco Dynabeads™ or Anti-Biotin MACSiBead™ Particles loaded with biotinylated human CD3 and CD28 antibodies. The methods may comprise transducing the cells with lentivirus. The methods may comprise growing the cells to between about 10×10⁶ cells and 250×10⁶ cells and delivering the treated cells to a subject.

In some embodiments, the cell therapy may be autologous. In such embodiments, the methods may comprise extracting cells for treatment from a subject. The methods may comprise delivering the treated cells to the subject.

In other embodiments, the cell therapy may be allogeneic. In such embodiments, the methods may comprise obtaining cells from a cell donor. In certain embodiments, the methods may comprise providing cells from a cell donor to a user. The methods may additionally comprise delivering the treated cells to a cell recipient.

In yet other embodiments, the cell therapy may be syngeneic. The methods may comprise obtaining or providing cells from a manufacturer. The methods may additionally comprise delivering the treated cells to a cell recipient.

In some embodiments, the methods may comprise introducing the cells into the perfusion chamber, optionally in an activated state. The cells may be concentrated in the perfusion chamber by a factor of 2, 5, 10, 25, or 50. The method may comprise concentrating the cells to a concentration of at least about 5×10⁶ cells/mL, at least about 20×10⁶ cells/mL, at least about 30×10⁶ cells/mL, at least about 50×10⁶ cells/mL, at least about 100×10⁶ cells/mL, or at least about 125×10⁶ cells/mL.

In certain embodiments, the cells introduced into the perfusion chamber, optionally in an activated state, may be in a high-density suspension. For instance, the cells introduced may be at a concentration of at least about 5×10⁶ cells/mL. The cells introduced may be at a concentration of at least about 20×10⁶ cells/mL, at least about 30×10⁶ cells/mL, at least about 50×10⁶ cells/mL, at least about 100×10⁶ cells/mL, or at least about 125×10⁶ cells/mL.

By introducing the cells at a concentration greater than about 20×10⁶ cells/mL or greater than about 30×10⁶ cells/mL (in an activated state or otherwise), the cell therapy method may reduce the time needed for sufficient cell expansion, thus reducing overall protocol time. In certain embodiments, introducing the cells at such a high density may eliminate the need for a cell expansion step.

By introducing the cells at a concentration greater than about 20×10⁶ cells/mL or greater than about 30×10⁶ cells/mL (in an activated state or otherwise), the cell therapy method may be performed without a transduction efficiency enhancing agent. Briefly, by increasing the density of cells and/or increasing the density of transducing agent, there is a greater probability of virus-cell interaction. Thus, the transduction efficiency may be substantially the same with a higher density cell suspension free of transduction efficiency enhancing agent, as with a lower density cell suspension including a transduction efficiency enhancing agent. The ability to perform high efficiency transduction without a transduction efficiency enhancing agent may reduce overall protocol time and cost. In some embodiment, the concentration of transducing agent (for example, viral vector) may be less than 150×10⁶ TU/mL, less than 80×10⁶ TU/mL, less than 40×10⁶ TU/mL, or less than 20×10⁶ TU/mL.

In some embodiments, the method may further comprise measuring at least one parameter of the cells or the media. The at least one parameter may be selected from pH, optical density, dissolved oxygen concentration, temperature, and light scattering. The method may comprise determining a state of the cells responsive to the measurement of the at least one parameter. The cell state may be associated with at least one of metabolic activity of the cells, average size of the cells, and density of the cells in the media.

In some embodiments, the method may comprise controlling the at least one measured parameter of the cells or media. The effective volume of the at least one additive may be selected responsive to the measured at least one parameter. As previously described, pH may be measured to determine metabolic activity of the cells. Responsive to a pH measurement, pH control may increase viability of the treated cells.

In certain embodiments, one or more sensor may be used to determine the at least one parameter measurement. A controller operatively connected to the sensor may generate a response to control the at least one parameter. The response may comprise administering the effective volume of at least one additive responsive to the measured at least one parameter.

Optical density and pH may be measured to determine progress of the activation cycle and timing of transduction and/or subsequent activation cycles. While not wishing to be bound by theory, cell activation typically follows a predictable growth profile. Upon activation, the diameter of the cells typically increases for 2-4 days (for example, from approximately 10 μm to approximately 12 μm), and then returns to the starting diameter (for example, approximately 10 μm) over the next 3-5 days. In the typical growth cycle, cells may proliferate for 7 to 10 days before becoming exhausted, triggering another round of activation to continue growth. The typical indicator for exhaustion is a reduction in growth rate. While cell size can be assayed by removing cells and measuring cell size in a microscope or flow cytometer, it is generally a manual process and labor intensive.

The methods described herein may comprise measuring a metabolic indicator, such as change in pH, oxygen consumption, growth rate, carbon dioxide production, lactate production, or glucose consumption, as an indicator for cell state, for example, cell activation and exhaustion. The methods may comprise determining the cell state to select the optimal time for cell treatment cycles, for example, transduction. The method may be used for autologous cell therapies. Each subject's cells may behave differently in response to cell activation. Efficiencies may be gained by determining the optimal time for treatment cycles through a measurement, as compared to, for example, a predetermined time.

Optical density and/or light scattering may be measured to determine the total cell density in the perfusion chamber. The growth rate of the cells can be determined from the slope of the total cell density curve. In some embodiments, an increase in growth rate at the beginning of activation may be used to initiate transduction. In some embodiments, a reduction in growth rate after cell activation may be used to initiate another activation cycle or harvest of the cells. In some embodiments a reduction in optical density after delivering activator to the culture may be used to initiate a completion of an activation cycle. Thus, the amount of time effective for cell activation may be determined responsive to a measured optical density and/or light scattering.

In some embodiments, the method may comprise determining timed delivery of the cell transducing agent responsive to a measurement of pH and/or calculation of added carbon dioxide gas or base as pH control agents. The method may comprise measuring the pH and calculating a quantity of added carbon dioxide gas or pH control agent (for example, base) to a perfusion chamber to maintain the media at a desired pH value. The method may comprise measuring pH and/or calculating the quantity of added carbon dioxide gas or pH control agent (for example, base) to the perfusion chamber to maintain the perfusion chamber at a desired pH value following addition of the cell activator for a period of time, for example, immediately to 5 days after introducing the cell activator. The method may comprise determining rate of change of added carbon dioxide or pH control agent (for example, base) to select a time to introduce the transducing agent. Responsive to observing a change in a rate of decrease in the quantity of added carbon dioxide gas or a change in the rate of increase of added base to the perfusion chamber after introducing the cell activator, the method may comprise introducing the transducing agent. Thus, addition of the transducing agent may be controlled responsive to measured pH and/or calculated addition of carbon dioxide gas or pH control agent in the perfusion chamber.

In another embodiment, the method may comprise determining timed delivery of the cell activator responsive to a measurement of optical density. The method may comprise measuring the rate of change of the density of cells after addition of the cell activator, for example, from immediately to 10 days after introducing the cell activator. Responsive to observing a decrease in the rate of change of the density of cells after the initial measurement, the method may comprise adding additional cell activator. Thus, the method may comprise controlling amount of cell activator responsive to a measured optical density.

Additionally, metabolic indicators, such as carbon dioxide supplementation rate or base/acid solution delivery rate for maintaining a pH setpoint may be measured as indicators for cell activation and exhaustion. By monitoring pH of the media, which may be controlled by the quantity of carbon dioxide or base/acid solution, the state of the cell activation and exhaustion cycle can be determined. In some embodiments, the method may comprise starting another activation cycle, finishing an activation cycle, finishing an expansion cycle, beginning a transduction cycle, and/or harvesting the cells may be performed responsive to the determined state of the cell activation and exhaustion cycle. Thus, the treatment protocol and/or period of time of each cycle may be selected responsive to a measured pH of the media. Similarly, the treatment protocol and/or period of time of each cycle may be selected responsive to a state of the cells determined from a measured dissolved oxygen concentration of the media.

Optical density and/or light scattering may be measured to determine the total cell density in the perfusion chamber. Cells may additionally be sensitive to fluctuations in temperature, pH, and dissolved oxygen concentration of the media. In some embodiments, viability of the cells may be maintained by controlling cell density in the perfusion chamber. Viability of the cells may be maintained by controlling temperature, dissolved oxygen concentration, and/or pH of the media for a known cell density.

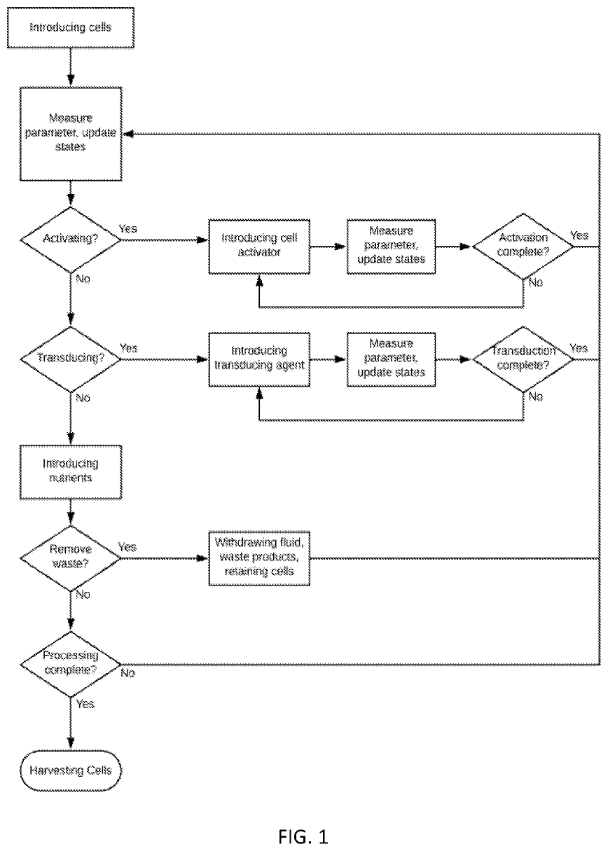

FIG. 1 is a flow diagram of an exemplary method of treating cells. Briefly, the exemplary method includes introducing cells into the perfusion chamber. The method includes introducing nutrients and/or cell media into the perfusion chamber and measuring at least one parameter. If the parameter is indicative of a desired cell concentration, viability, and/or metabolic activity, the method includes introducing an additive comprising a cell activator and measuring at least one parameter. If the parameter is indicative of a desired cell activation and/or expansion rate, the method includes introducing an additive comprising a transducing agent and measuring at least one parameter. If the parameter is indicative of a desired transduction efficiency, the method includes expanding the cells and measuring at least one parameter. If the parameter is indicative of the desired cell number, the method includes harvesting the cells. The method may include withdrawing cell waste and byproducts from the perfusion chamber at any point during treatment, or continuously, if necessary. The method may comprise repeated cell activations, transductions, and/or expansions. In some embodiments, the method may comprise introducing the cell activator, introducing the transducing agent, and measuring at least one parameter. If the parameter is indicative of a desired cell activation and/or expansion rate, the method may include harvesting the cells. If the parameter is not indicative of a desired cell activation and/or expansion rate, the method may include repeating the cell activation cycle.

In other embodiments, a predetermined period of time may elapse to determine when to continue to the next cycle. In yet other embodiments, the method may include using historical data of one or more of the measured parameters to learn and predict the period of time between cycles.

In accordance with another aspect, there is provided a system for performing cell culture. The system may comprise a perfusion chamber. The perfusion chamber may be suitable for performing the methods described herein. The perfusion chamber may be formed or lined with a material inert to the cells and cell treatment additives disclosed herein. The system and/or perfusion chamber may have one or more embodiments as described in any one or more of U.S. Pat. No. 9,328,962 titled “Apparatus and methods to operate a microreactor,” filed on Jan. 25, 2013; U.S. Patent Application Publication No. 2014/0234954 titled “Methods and apparatus for independent control of product and reactant concentrations,” filed on Feb. 14, 2014; U.S. Pat. No. 9,176,060 titled “Apparatus and methods to measure optical density,” filed on Apr. 9, 2012; and U.S. Pat. No. 9,248,421 titled “Parallel integrated bioreactor device and method,” filed on Oct. 10, 2006, each of which is herein incorporated by reference in their entireties for all purposes.

The perfusion chamber may generally have an inlet fluidly connectable to a source of cells to be treated and an outlet fluidly connectable to a waste chamber. An additional outlet may be fluidly connectable to a harvest receptacle. The perfusion chamber may have a predetermined internal volume. In certain embodiments, the internal volume may be about 100 mL or less, for example, about 50 mL or less. The internal volume may be between about 1 mL and about 5 mL, between about 2 mL and about 10 mL, between about 2 mL and about 20 mL, between about 5 mL and about 20 mL, between about 10 mL and about 30 mL, or between about 20 mL and about 50 mL. The internal volume may be less than about 30 mL, less than about 20 mL, less than about 10 mL, less than about 5 mL, less than about 4 mL, less than about 3 mL, less than about 2.5 mL, less than about 2 mL, or less than about 1 mL.

The perfusion chamber may be configured to reversibly substantially isolate the contents of the perfusion chamber from the environment. For instance, the perfusion chamber may comprise valves positioned at the inlet and/or outlet of the perfusion chamber configured to control fluid flow. The perfusion chamber may comprise valves positioned at the inlet and/or outlet of the perfusion chamber configured to control fluid flow, for example, rate of fluid flow. The perfusion chamber may be hermetically sealed when the valves are closed. In certain embodiments, the valves may be pneumatically actuated valves.

The system may comprise a filter membrane within or downstream from the perfusion chamber. The filter membrane may have pores sized to concentrate a desired component within the perfusion chamber. For example, the filter membrane may have pores sized to concentrate cells within the perfusion chamber and allow passage of smaller particles. Such a filter membrane may have an average pore size of between 0.2 μm and about 50 μm, for example, between 1 μm and about 20 μm or between 1 μm and about 10 μm. The average pore size may be selected based on the target cell. The filter membrane may have pores sized to concentrate the cell activator within the perfusion chamber. Such a filter membrane may have an average pore size of between 1 nm and 20 nm, for example between 1 nm and 10 nm. The filter membrane may have pores sized to concentrate the transducing agent within the perfusion chamber. Such a filter membrane may have an average pore size of between 10 nm and 200 nm, for example, between 10 nm and 100 nm. The average pore size may refer to an average pore size of at least 80% of the pores, at least 90% of the pores, or at least 99% of the pores. In general, the filter membrane may have pores sized to concentrate cells within the perfusion chamber while allowing passage of cell waste and byproducts. The filter membrane may be formed of a substantially inert material.

In certain circumstances, additives, transducing agents, and/or cells may cause filter clogging. Filter membrane pore sizes may be selected to minimize filter retention and clogging. For example, average filter membrane pore sizes of 1.2 μm or larger, but smaller than an average size of the target cell (for example, about 10 μm) may be used to minimize filter clogging by the transducing agent (for example, lentivirus). In the exemplary embodiments, media without transducing agent may be perfused through the larger filters to retain cells but allow the transducing agent to be washed out and diluted. Integration of the transducing agent removal filter directly into the perfusion chamber may allow automation of the transduction process.

In certain embodiments, the system may comprise a plurality of filters each having a different average pore size. Briefly, maintaining a high concentration of the transducing agent, while perfusing fresh nutrients and removing cell waste and byproducts may require a high perfusion rate and a high concentration of transducing agent in the feed stream. The perfusion chamber may include and be operated with two or more filters, fluidically connected to the perfusion chamber or integrated directly into the perfusion chamber, that retain different size particles or additives. The concentration of additives within the perfusion chamber may be varied independently of the concentration of additives in the feed streams.

In one exemplary embodiment, the system may comprise a first filter membrane having an average pore sized to retain transducing agent while passing small molecules. The same system may comprise a second filter membrane having an average pore sized to retain cells while passing the transducing agent. With such a system, the transducing agent concentration may be increased by perfusion through the first filter to provide nutrients to a high density of cells, while transducing agent is introduced. The first filter may have a pore size less than 0.2 μm. The second filter may have a pore size greater than 0.2 μm. The filters may selectively concentrate, retain, and dilute lentiviral vectors (as an exemplary transducing agent) from the cell-holding chamber. When the transduction operation is satisfactorily completed, perfusion may proceed through the second filter that passes the transducing agent to wash the transducing agent from the culture chamber. These embodiments are exemplary. Other embodiments including a plurality of filters are within the scope of the disclosure.

In embodiments in which porous filter membrane are not used, any other filter-free methods of cell retention and/or separation may be used to retain cells and wash out additives. In some embodiments, filter free methods may be integrated into the system to enable the automation of the process.

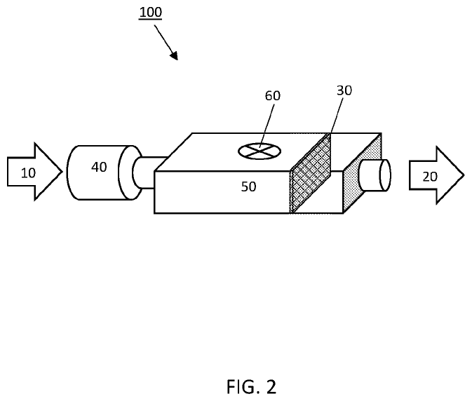

An exemplary perfusion chamber 100 is shown in FIG. 2. The exemplary perfusion chamber 100 includes at least one inlet 10, at least one outlet 20, at least one filter 30, and internal chamber 50. The exemplary perfusion chamber 100 includes at least one check valve 40, which may be a pneumatic valve, positioned at the at least one inlet 10 to substantially isolate the contents of the internal chamber 50 when actuated. The exemplary perfusion chamber 100 includes at least one port 60 for fluid communication with the internal chamber 50. The at least one port 60 may be used as an access port for a sensor. As previously described, the perfusion chamber may comprise a plurality of inlets 10, outlets 20, filters 30, valves 40, and ports 60 as necessary.

The system may comprise a source of cells fluidly connectable, and in use fluidly connected, to the perfusion chamber. The cells may be suspended in a media, for example, a cell culture media. The media may comprise one or more nutrient or additive in an amount effective to maintain viability of the cells. The source of the cells may comprise any cells and/or cell density as previously described.

The system may comprise a source of an additive fluidly connectable, and in use fluidly connected, to the perfusion chamber. The additive may be in aqueous, particle, or gel form. The additive may be in any form suitable for combination with the cells within the perfusion chamber. In exemplary embodiments, the additive may comprise one or more of cell culture media, a transducing agent, a pH control agent, and a cell activator. In general, any nutrient, agent, or additive disclosed herein may be fluidly connectable or connected to the perfusion chamber. For embodiments comprising more than one additive fluidly connectable to the perfusion chamber, each additive may be independently fluidly connectable or connected to the perfusion chamber. In other embodiments, one or more additives may be combined, and the combination may be fluidly connectable or connected to the perfusion chamber.

The system may comprise at least one sensor selected from a pH sensor, an optical density sensor, a dissolved oxygen sensor, a temperature sensor, and a light scattering sensor fluidly connected to the perfusion chamber. Thus, the at least one sensor may be configured to measure at least one parameter of the cells or the media selected from pH, optical density, dissolved oxygen concentration, temperature, and light scattering, respectively. The at least one sensor may be an in-line sensor positioned at an inlet or outlet of the perfusion chamber. The at least one sensor may be positioned at least partially within the perfusion chamber. Any sensor positioned partially within the perfusion chamber may be introduced through an otherwise hermetically sealed inlet or integrated into the perfusion chamber.

The system may comprise a controller. The controller may be configured to direct the cells and/or additives into the perfusion chamber and/or the cell waste and byproducts out of the perfusion chamber. The controller may be operatively connected to one or more pumps or valves to effectively direct the fluids within the system. The controller may be configured to direct the additive into the perfusion chamber at a flow rate as previously described. The controller may be configured to maintain a selected concentration of one or more additive within the perfusion chamber.

In some embodiments, the controller may be operatively connected to the at least one sensor. The controller may be configured to direct an effective volume form the source of the cells and/or the source of the additive into the perfusion chamber responsive to a measurement obtained from the at least one sensor. In certain embodiments, the controller may be configured to maintain a target pH value, as previously described. In some embodiments, the controller may be configured to initiate a cycle of treatment upon indication that a previous cycle has operated to completion or substantial completion.

The controller may be a computer or mobile device. The controller may comprise a touch pad or other operating interface. For example, the controller may be operated through a keyboard, touch screen, track pad, and/or mouse. The controller may be configured to run software on an operating system known to one of ordinary skill in the art. The controller may be electrically connected to a power source. The controller may be digitally connected to the one or more components. The controller may be connected to the one or more components through a wireless connection. For example, the controller may be connected through wireless local area networking (WLAN) or short-wavelength ultra-high frequency (UHF) radio waves. The controller may further be operably connected to any pump or valve within the system, for example, to enable the controller to direct fluids or additives as needed. The controller may be coupled to a memory storing device or cloud-based memory storage.

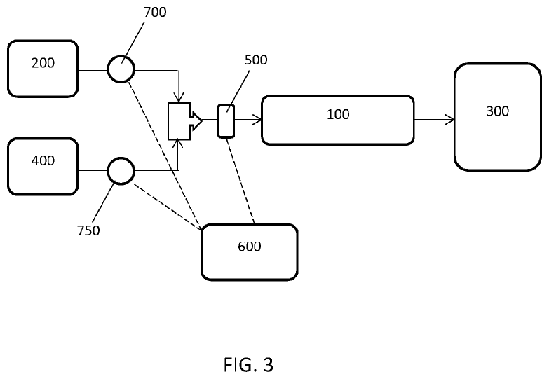

An exemplary system for treating cells 1000 is shown in FIG. 3. The exemplary system 1000 includes a perfusion chamber 100 as shown in FIG. 2. The perfusion chamber 100 is fluidly connected to a source of cells 200 and a waste chamber 300. The perfusion chamber 100 is fluidly connected to at least one source of an additive 400. The system includes at least one sensor 500. While sensor 500 is shown positioned and configured to measure a parameter of the suspension upstream from the perfusion chamber 100, it should be understood that the system 1000 may include a plurality of sensors 500 and/or the sensor 500 may be positioned and configured to measure a parameter of the suspension within the perfusion chamber 100, upstream from the perfusion chamber 100, and/or downstream from the perfusion chamber 100. The system 1000 includes controller 600 operatively connected to the at least one sensor 500. The system 1000 includes pump 700 positioned and configured to direct cells in media from the source of cells 200 to the perfusion chamber 100. The system 1000 includes pump 750 positioned and configured to direct additive from the source of the additive 400 to the perfusion chamber 100. Pumps 700, 750 may be operatively connected to the controller 600.

In accordance with another aspect, there is provided a method of facilitating cell therapy. The method may comprise providing one or more components of a system for performing cell culture, as previously described. For example, the method may comprise providing a perfusion chamber, at least one sensor, and/or a controller. The method may comprise instructing a user to operatively connect the controller to the at least one sensor and/or to one or more valves or pumps within the system configured to direct fluids. The method additionally may comprise instructing a user or operator to fluidly connect the perfusion chamber to a source of cells and/or a source of an additive, as previously described.

In certain embodiments, the method may comprise programming the controller to operate in accordance with selected parameters. For instance, the method may comprise instructing the user to select a working range of at least one parameter selected from pH, optical density, and light scattering and program the controller to direct the effective volume of the additive responsive to the at least one selected working range.

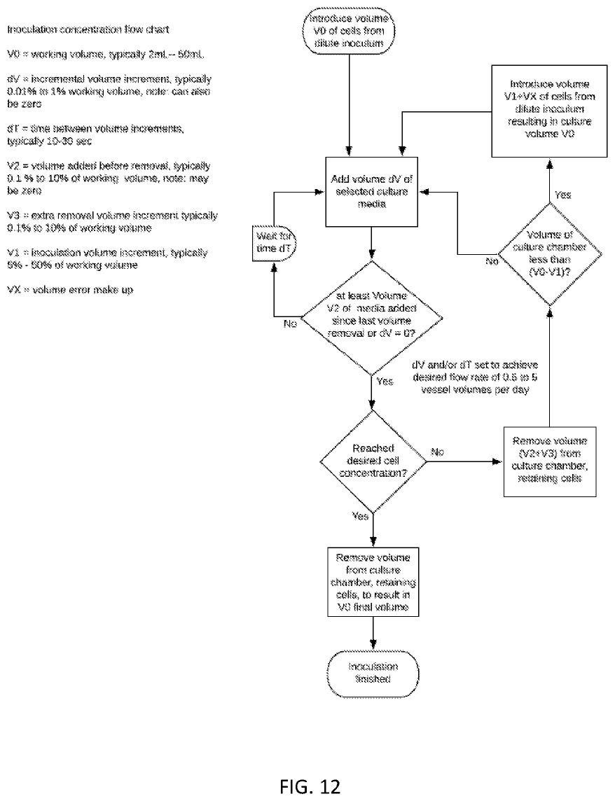

The method may comprise treating cells as shown in the exemplary flow diagrams of FIGS. 12-17. In certain embodiments, a controller may be programmed to operate a cell treatment system consistently with the flow diagrams of FIGS. 12-17. Thus, the methods may comprise programming a controller to generate one or more instructions as shown in FIGS. 12-17. Multiple controllers may be programmed to work together to operate the system.

In other embodiments, one or more of the flow diagram processes from FIGS. 12-17 may be manually or semi-automatically executed.

As shown in FIG. 12, the method may comprise inoculating a perfusion chamber with a media comprising cells and optionally concentrating the cells within the perfusion chamber. Briefly, the method may comprise introducing a volume of cells from a source inoculum. The method may comprise continuously perfusing at least one additive by adding a volume of the at least one additive. The method may comprise determining if a desired cell concentration has been reached. If the desired cell concentration has not been reached, the method may comprise removing a volume of fluid from the perfusion chamber, larger than the volume of additive previously added, retaining cells, and, optionally, introducing an additional volume of cells from the source inoculum. If the desired cell concentration has been reached, the method may comprise removing media from the culture chamber to complete the inoculum. The concentrations, volumes, and working times shown in FIG. 12 are exemplary.

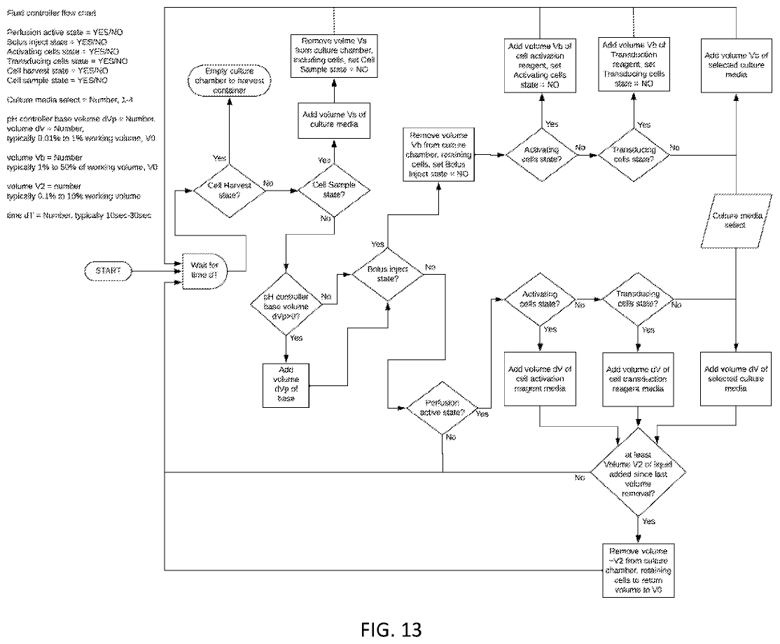

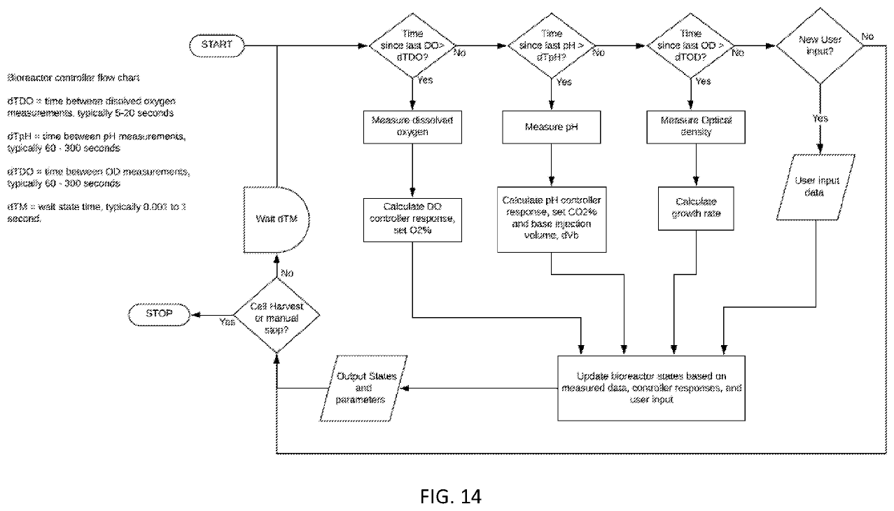

As shown in FIG. 13, the method may comprise controlling the flow of fluid into and out of the perfusion chamber based on state variables and process variables. The flow chart of FIG. 13 may be executed by a fluid controller. Thus, in some embodiments, the state variables and process variables may be determined by a process flow operating on a bioreactor controller (as shown, for example, in FIGS. 14-16). Depending on the value of the state variables and process variables, volumes of selected fluids such as various culture media, cell activation reagents, or cell transduction reagents may be added, and removed. The concentrations, volumes, state variables, and working times shown in FIG. 13 are exemplary.

As shown in FIG. 13, the method may comprise fluid flow through bolus additions where a volume of fluid, retaining cells, may be removed from the culture chamber. The removed volume may be replaced by a selected media as a bolus. Alternatively, the bolus may first be added and then fluid removed. The method may comprise fluid flow through continuous perfusion where small incremental volumes of a selected fluid may be added to the culture chamber periodically. The period may range from a few seconds to a few hours. Periodically volumes of fluid may be removed, retaining cells within the culture chamber. Volume removal may be triggered by the number of small incremental volumes added, ranging from 1 to 1000 or 10 to 100 or 100 to 1000 incremental volumes. The relatively frequent volume additions and removals may effectively provide a continuous flow.

The method may comprise addition of a pH control agent (for example, a base, such as sodium carbonate, sodium bicarbonate, ammonium hydroxide, sodium hydroxide, or other base) responsive to a pH measurement and calculation of a pH controller response. The method may comprise removing a cell sample by adding a volume Vs, of a selected culture media and then removing the volume Vs from the perfusion chamber, including cells in the sample. The volume of sample may range from 1% to 10% of the working volume or 10% to 50% of the working volume.

The method may comprise harvesting the cells. For instance, during harvesting the cells, the entire contents of the perfusion chamber may be removed, collecting all of the cells. Harvesting the cells may additionally comprise washing the emptied perfusion chamber with additional media to collect remaining cells. The method may comprise selecting fluids to introduce into the culture chamber based on the state variables.

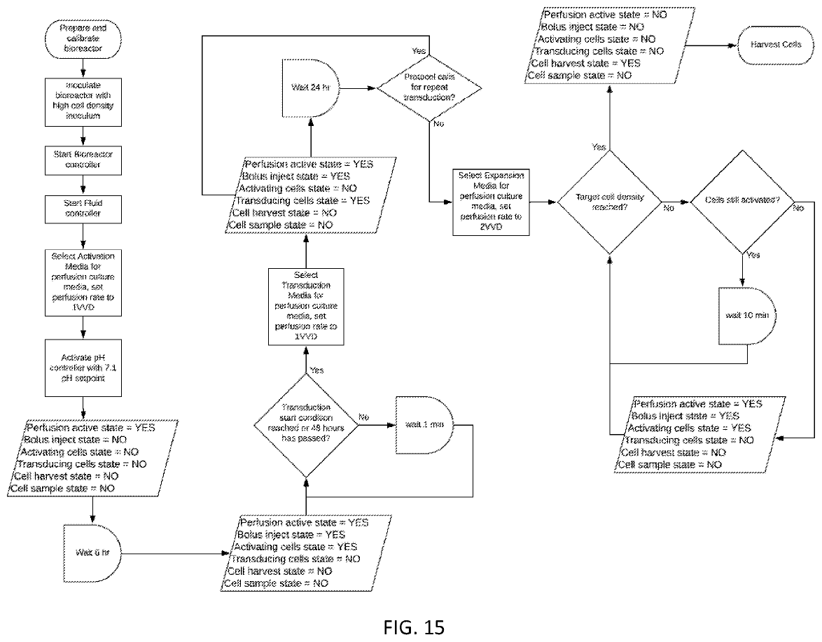

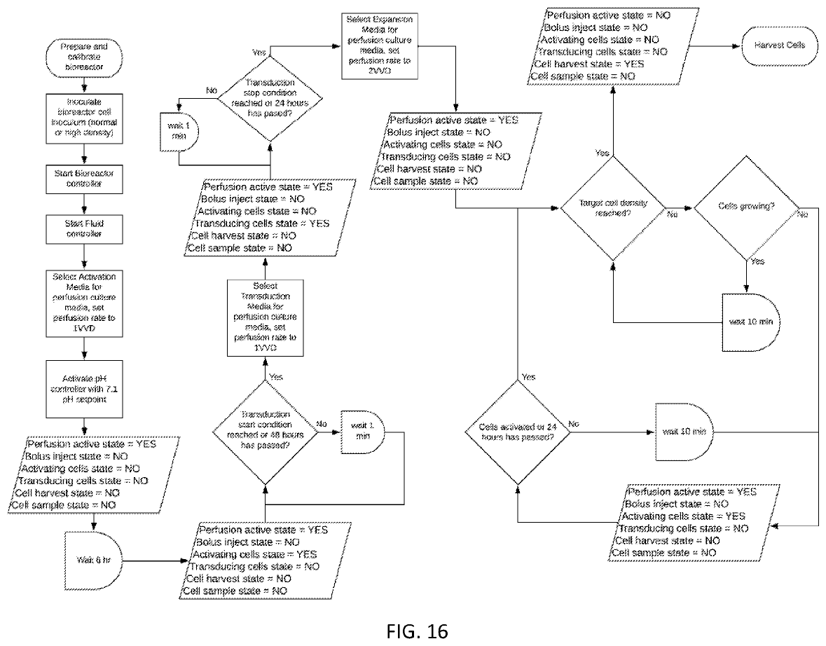

As shown in FIG. 14, the method may comprise treating cells responsive to a measurement of dissolved oxygen, pH, or optical density. The flow chart of FIG. 14 may be executed by a bioreactor controller. Thus, in some embodiments, the method may comprise treating cells responsive to calculated controller or derived parameters (such as growth rate), user input, and a process flow program (for example, as shown in FIGS. 15-16). Briefly, the method may comprise periodically measuring at least one of dissolved oxygen, pH, and optical density. The method may comprise determining a cell state based on the measured parameter. The method may comprise determining an output state, parameters such as volumes for the fluid flow controller, or transition conditions for process flow programs, based on the measured parameters. The method may comprise providing user input data and updating a response protocol based on the user input data. The method may comprise determining whether the cells are ready for harvest based on the measured parameter and the user input data. The working times shown in FIG. 14 are exemplary.

As shown in FIG. 15, which is a flow chart of a process flow program utilizing bolus additions of cell activator and cell transduction reagent, the method may comprise treating cells based on a bolus activation and transduction protocol. Briefly, the method may comprise inoculating the perfusion chamber with a high-density cell suspension. The method may comprise perfusing a media to prepare cells for activation. The method may comprise waiting a period of time before introducing a bolus of cell activation reagent. The method may comprise waiting a period of time until transduction start conditions are reached or detected. The method may comprise introducing a first volume of transducing agent. The method may comprise waiting a period of time and determining whether a second volume of transducing agent will be introduced. The method may comprise introducing a second volume of transducing agent. The method may comprise introducing expansion media and determining whether target cell density has been reached. The method may comprise determining whether cells are still activated. The method may comprise introducing an additional bolus of cell activation reagent. If conditions are met, the method may comprise harvesting the cells. The set points, selected media, flow rates, and working times shown in FIG. 15 are exemplary.

As shown in FIG. 16, which is a flow chart of a process flow program utilizing perfusion of cell activation reagent and transduction reagent, the method may comprise treating cells based on a perfusion activation and transduction protocol. Briefly, the method may comprise inoculating the perfusion chamber with a cell suspension. The method may comprise treating cells with perfusion of a media optimized for cell activation. The method may comprise waiting a period of time and then perfusing with media including a cell activation reagent. The method may comprise waiting a period of time until transduction start conditions are reached or detected. The method may comprise introducing media comprising the transducing agent continuously until a transduction stop condition has been reached or detected. The method may comprise introducing expansion media or perfusion culture media until a target cell density has been reached. The method may comprise determining whether cells are growing and re-activating the cells, waiting a period of time until cell activation has been reached. If the target cell density is reached, the method may comprise harvesting the cells. The set points, selected media, flow rates, and working times shown in FIG. 16 are exemplary.

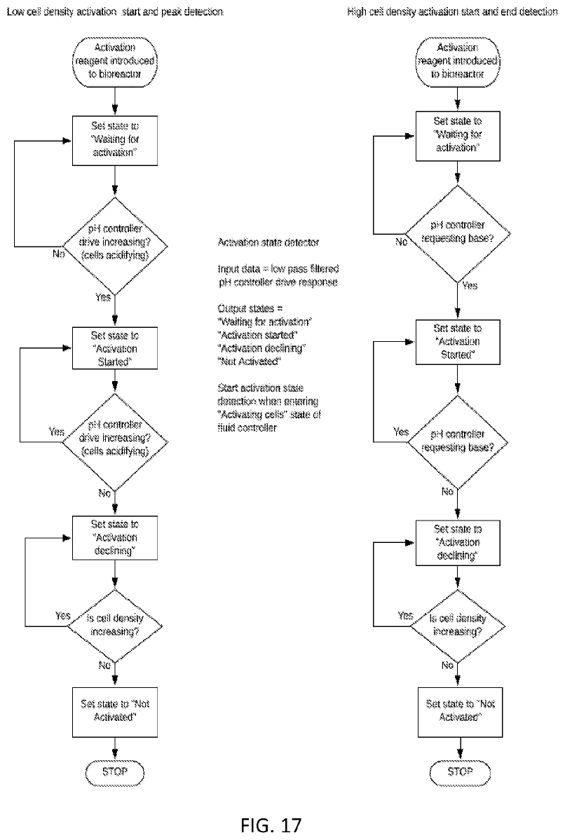

As shown in FIG. 17, which are flow charts for detecting the activation state of cells, the method may comprise detecting the cell activation state through measurements of pH and cell density. Briefly, for low cell density activation detection, the method may comprise waiting for a pH measurement to indicate a cell state associated with cells waiting for activation. In some embodiments, a pH controller drive may be used to signal an increase in a “Waiting for activation” state. When the pH controller drive signal increases, signifying cells are acidifying, the media and cells may be activated. The activation detector may enter an “Activation started” state. The method may comprise waiting until the pH controller drive signal does not increase for the activation detector to enter an “Activation declining” state.

The method may comprise monitoring the cell density, through optical density measurements, for example, to determine if cells are growing. If not, then the activation detector enters a “Not Activated” state. The method may also comprise an activation state detector for high cell density activation, where whether the pH controller requests base is the signal for switching between the “Waiting for activation”, “Activation started”, and “Activation declining” states. The state of the activation detector may be an input to other processes, such as deciding wither to start transduction or whether to initiate an additional activation. The conditions for switching states shown in FIG. 17 are exemplary.

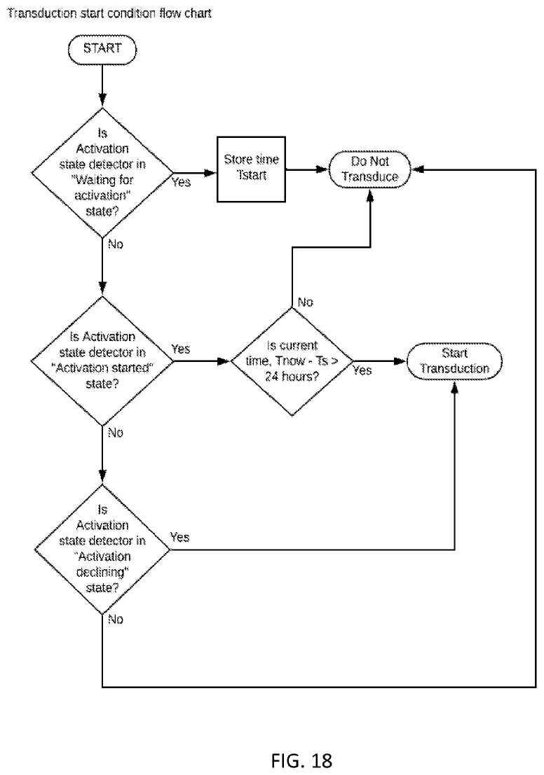

As shown in FIG. 18, which is a flow chart describing detecting an exemplary transduction start condition, the method may comprise deciding when to start transduction based on an estimated activation state of the cells. Briefly, the method may comprise checking the activation detector state, and time, and returning a “Do not transduce” or “Start transduction” directive. The method may comprise returning a “Do not transduce” directive when the activation detector is in a “Waiting for activation” state or “Not activated” state, or if the activation detector is in an “Activation started” state for less than 24 hours. The method may comprise returning a “Start transduction” directive if the activation detector is in an “Activation declining” state, or if the activation detector is in an “Activation started” state for more than 24 hours. The times and conditions shown in FIG. 18 are exemplary.

In some embodiments, the method may comprise providing the source of the cells and/or the source of the additive. The source of the cells and/or the source of the additive may be a vessel or chamber fluidly connectable to the perfusion chamber. In certain embodiments, the method may comprise providing the cells and/or one or more additive. Thus, in certain embodiments, a kit comprising the system, at least one additive, and instructions for use may be provided. In some embodiments, the kit may additionally comprise cells.

### EXAMPLES

The function and advantages of these and other embodiments can be better understood from the following examples. These examples are intended to be illustrative in nature and are not considered to be limiting the scope of the invention.

### Prophetic Example 1: Cell Therapy Method

In one embodiment of a cell therapy method, selected cells harvested from a subject are introduced into a perfusion chamber having a volume less than or equal to 50 mL. A transducing agent (for example, a retroviral vector, gamma retroviral vector, alpha retroviral vector, lentiviral vector, transposon, or mRNA electroporation), cell culture media, a pH control agent (for example, a sodium hydroxide, sodium carbonate, sodium bicarbonate, ammonia, potassium hydroxide, carbon dioxide, hydrochloric acid, and phosphoric acid), and a T-cell activator (for example, magnetic beads, particle-bound antibodies, antigen presenting cells) are introduced in an automated protocol into the perfusion chamber.

After inoculation the subject's cells in the perfusion chamber, the T-cell activator may be delivered into the perfusion chamber via bolus injection or perfusion with culture media. Then the transducing agent may be delivered to the perfusion chamber via bolus injection or perfusion with culture media and the perfusion chamber may be agitated to increase shear and promote transduction. After effective transduction of a target percentage of cells media may be exchanged through the perfusion filter to wash out the transducing agent. Cells may be expanded in the perfusion chamber under perfusion conditions. After the end of the cell activation cycle cells may be harvested for formulation.

If more cells are required, cells may undergo a second activation cycle, either through perfusion or bolus injection of activator. The cell activation may be performed at the end of the first expansion cycle. Typically, the second activation cycle starts at higher cell density because cells have been expanded. The perfusion of the cell activator may be performed at a concentration effective to deliver total concentrations of activator proportional to the higher cell density. Conventional activator solutions have a starting density appropriate only for low cell densities (<2×10⁶ cells/mL). For most cell activators, if more activator is mixed with media to reach the desired quantities of activator appropriate for the higher cell density, the media may be diluted too much and may be unable to support the higher cell density.

Active pH control may be performed for higher cell density perfusion, either through high perfusion rate (>3 vvd) or addition of a basic pH control fluid to handle the acidification resulting from activated T-cells. After delivery of the cell activator, media perfusion may be performed until the second expansion cycle is complete. If subsequent expansions are required, the process can be repeated as many times as necessary. After a treatment appropriate cell number is reached, cells may be ejected into a sterile storage container and cooled or frozen as necessary.

### Prophetic Example 2: Small Volume, High-Density Cell Therapy Methods

By utilizing a small perfusion microbioreactor for a gene modified cell therapy such as CAR-T production, the culture environment can be rapidly controlled and changed to meet the needs of the growing cells. It is contemplated that using a cell culture chamber volume less than 50 mL, for example, 20 mL, 10 mL, 5 mL, or 2 mL, volumes and quantity of media, growth factor, transducing agents, and cell activators can be significantly reduced, increasing ease of use and having large cost savings. At small volumes, expanding enough cells for final treatment may require starting treatment at high cell densities from subjects, for example, 5×10⁶ to 100×10⁶ cells/mL. The higher cell density suspension may be inoculated into the perfusion chamber. It is contemplated that perfusion with fast media exchange may provide better results. It is further contemplated that the small volume perfusion chamber may enable transduction at high densities of transducing agent, while still maintaining low total quantities of transducing agent. The higher density of transducing agent is generally not possible in larger volume reactors (>100 mL).

In certain embodiments, if a sufficient volume of cells is obtained from the source of the cells (for example, harvested from the subject), it is contemplated that all the cells may be transduced and directly harvested, skipping expansion and reducing manufacturing time significantly. The methods may include controlling the cell culture environment carefully at the high cell density. Such methods may significantly reduce the cost of cell therapy treatment by reducing the concentration of transducing agent required. If transducing agent is not available in a high enough concentration, it is contemplated that transducing agent retention filters for example, having a typical pore size of 0.2 μm or smaller can be used to concentrate the transducing agent and deliver an effective amount to the cells through perfusion.

For high density cultures in small volume rectors with fluid mixing, it is contemplated that a significantly smaller concentration of virus particles per cell may be sufficient (for example, up to less than 50% of the typical concentration of standard protocols) to transduce cells. The high density of cells may result in a high concentration of virus particles even at low quantities of virus particles per cell. Additionally, shear flows may be generated from small volume mixing. Interactions between cells and viruses may generally increase, and transduction efficiency may be improved.

### Example 1: Automated Cell Treatment for Gene Modified Cell Therapy

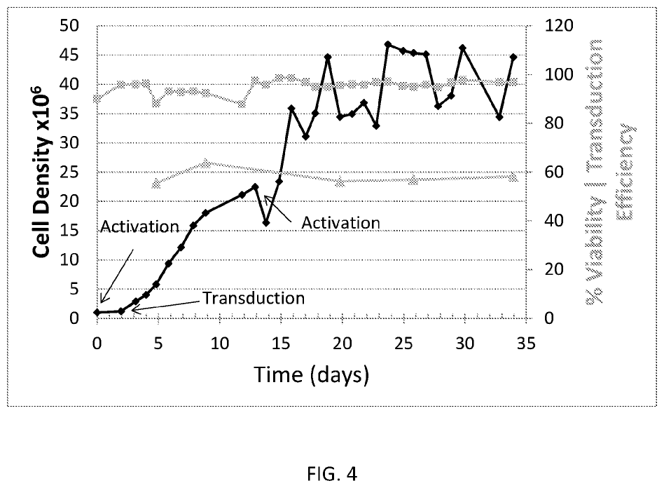

Conventional methods were used to produce gene modified T-cells analogous to a Chimeric Antigen Receptor-Modified T-cell (CAR-T cell) therapy. CAR-T therapy may be used to treat certain cancers. All unit operations were performed in situ. The results are shown in the graph of FIG. 4. The results in FIG. 4 correlate with a typical automated process for T-cell activation, transduction, and expansion in a perfusion chamber.

Briefly, T-cells were prepared (by the methods described in more detail in Example 9) and inoculated into the perfusion chamber at a cell density of 1M cells/mL. After two days, the cell density was 1.18M cells/mL and lentiviral vector with a GFP payload was introduced into the perfusion chamber by first removing 1 mL of cell-free media from the perfusion chamber and then filling with a mixture of 500 μL of viral vector solution and 500 μL of culture media. The quantity of virus was 125M infectious particles for a multiplicity of infection of 53. On day 12 when the cell density was 21M cells/mL, perfusion at 3 volumes per day of 2 mL TransACT™ in 22 mL of TexMACS™ was started and lasted for two days to provide an additional activation. Daily samples were removed from the perfusion chamber for cell count and viability measurement. Additional samples for transduction efficiency and vector copy number assays were taken on days 4, 8, 20, and 26.

Following a conventional low cell density protocol, the method of cell treatment in a small volume perfusion chamber performed similarly to conventional T-cell production processes. Transduction efficiency was approximately 50% and cell density reached 18M cells/mL with 92% viability after 9 days.

To assess high cell density performance, an additional activation was performed and high cell densities up to 45M cells/mL with 95% viability were achieved after 19 days.

The results demonstrate successful activation, transduction, and expansion of Human T-cells within a 2 mL working volume perfusion chamber, similarly to conventional T-cell processes with low cell density inoculum. Similar experiments may be repeated to assess the highest cell density achievable. It is expected that similar performance may be obtained with cell densities up to 100M cells/mL or 300M cells/mL.

### Example 2: High Density T-cell Activation, Transduction, and Expansion

Purified T cells, apheresis product, or peripheral blood mononuclear cells (PBMC) were inoculated into a small volume perfusion chamber, as described herein. Either the initial inoculum included an activator to stimulate T-cells (for example, magnetic Dynabeads™ or TransACT™) or the activator was introduced into the perfusion chamber through an input fluid port, either through perfusion or through bolus injection.

To obtain the data shown in FIG. 4, the cells were cultured in cell culture media including TexMACS™ T-cell culture media supplemented with 100 U/mL of IL-2. The cells were activated with TransACT™ conjugated with humanized CD3 and CD28 agonist at a ratio of 1:17. This nanomatrix particle size of the cell activator is smaller than the typical perfusion filter pore size, which allowed for removal of the cell activator by media exchange. Tested filters had an average pore size of from 0.2 μm to 1.2 μm. No magnetic separation was needed. Cells were stimulated at inoculation by combining TexMACS™ media, IL-2,and TransACT™ with cells to generate a 0.9×10⁶ cells/mL inoculum and 2 mL total volume of inoculum was injected into the perfusion chamber.

Transduction was performed up to 2 days post activation. On day 2 the cells were transduced with a protocol that utilized the perfusion filter rather than typical centrifugation. However, any integrated cell retention device may be used, such as a spin filter, acoustic cell separator, centrifuge, or cell sorter. First 500 μL of media was removed from the perfusion chamber through the outlet in preparation for lentivirus injection. Then 500 μL of media containing lentivirus was injected into the perfusion chamber. In this case, the concentration of lentivirus was appropriate for transduction with a bolus injection. If the concentration of the transducing agent is too dilute, the agent could be perfused into the perfusion chamber with a retention filter to concentrate the transducing agent without affecting the growth media composition. Filters having an average pore size of 0.2 μm or smaller may be used for lentivirus retention. Cells were then grown in batch for 24 to 48 hours to allow for transduction, followed by perfusion at 1 VVD or higher to wash out virus from the perfusion chamber. Perfusion was gradually increased to 3 VVD on day 8 in proportion to the increasing viable cell density.

As cell activation started to decay, a second round of activation was performed through perfusion or bolus injection, again depending on the total cell density and concentration of transducing agent. On day 13, media containing TransACT™ in TexMACS™ media at a ratio of 1:15, respectively, supplemented with 200 U/mL of IL-2, was perfused into the perfusion chamber at 3 VVD for 2 days. Typically, the concentration of TransACT™ for a second or subsequent round of activation per cell may be substantially the same as the initial round. However, the concentration of TransACT™ available did not allow for such high mixing ratios while still maintaining a proper media composition. By introducing the TransACT™ activator through perfusion, a much higher total concentration of activator was delivered to the cells. A total of 0.8 mL TransACT™ was delivered into the perfusion chamber by the end of 2 days.

Activation of T-cells with TransACT™ caused visible cell aggregation when introduced at high cell density. The cell aggregation was visualized by the drop-in cell density measured 24 hours post-activation on day 14. In addition, once flow of TransACT™ was removed, mixing and shear inside the perfusion chamber due to high perfusion rate caused separation of aggregated cells, resulting in a spike in cell density 24 hours after stopping TransACT™ flow. The second round of activation resulted in a noticeable increase in cell density of 2.5x over the next 7 days.

### Example 3: Growth Curves for High Density T-cells

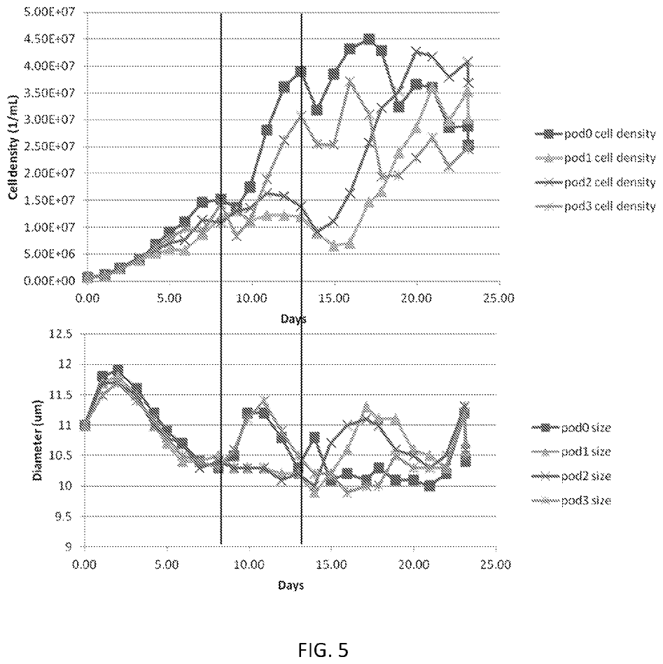

FIG. 5 shows growth curves for four simultaneous perfusion cultures. Briefly, T-cells were prepared by stimulating PBMC with TransACT™ and expanding in a G-rex culture flask. Inoculum was prepared using TexMACS™ media, 100 U/mL, IL-2, and TransACT™ cell activator with an inoculum density of approximately 10⁶ cells/mL. Re-stimulation was performed by introducing additional cell activator at day 8 for pod0 and pod3 and at day 13 for all pods. Cell diameter increase was correlated with T cell activation and expansion. For pod0 and pod3, the second re-stimulation did not result in substantial growth. Further tests may be performed to determined activation protocol for additional increase in cell density.

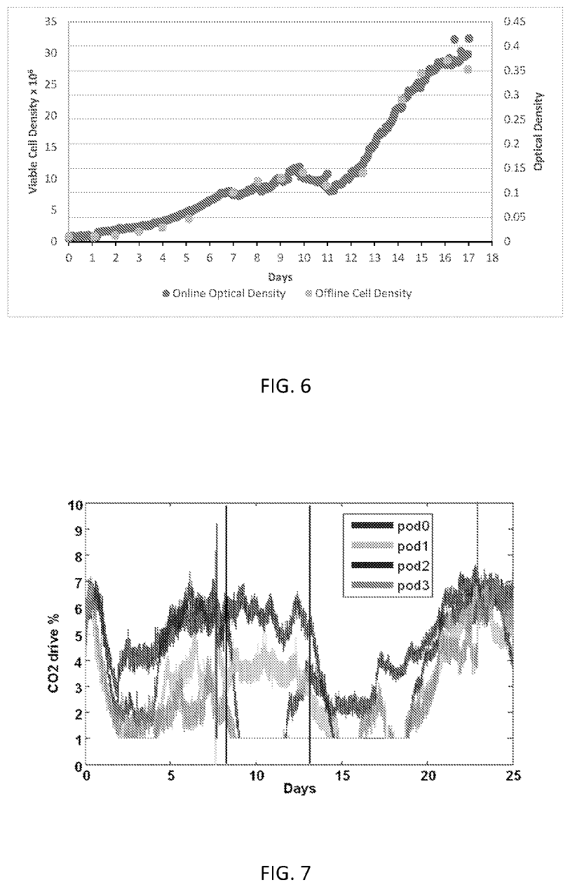

The data presented in FIG. 5 shows the behavior of cell size over the course of the growth. As expected, cell size was correlated with cell activity in response to the cell activator (here, TransACT™). As cells responded to the cell activator, cell size increased and cells started to divide. During the next 7 days, cell diameter slowly returned to its smaller size representative of dormant T-cells. A second round of activation again caused increase in cell size and gradual decrease again over the course of a few days. The reduction in cell diameter was also correlated with a reduction in growth rate, which can be seen in the graph of FIG. 6.

FIG. 6 is a graph of optical density over time. Optical density was measured online for the cell suspension. A visible decrease in growth rate was seen between day 7 and day 10. Optical density measurement was correlated with cell density. Another round of activation may be performed responsive to the measured decrease in growth rate. After a second cycle of activation on day 10, the optical density started to decrease. It is theorized that the optical density decrease was due to clumping from activator binding. Optical density measurement, through changes in growth rate was also correlated with the extent of cell activation.

Thus, optical density may be measured to determine cell activation(increase in growth rate) and substantial completion of cell activation (decrease in optical density).

FIG. 5 shows cell density growth curves and cell diameter data for four different cell cultures. A second cell activation cycle is highlighted, in which additional T-cell activation reagent was perfused along with fresh media. The data show the correlation between increased cell diameter and activation. FIG. 6 shows online optical density measurements that are correlated to cell density.

The data presented in FIGS. 5-6 shows how online sensor measurements for cell size and optical density can be correlated to the metabolic activity of cells, which enables monitoring and control over operations that impact cell metabolism, such as cell activation.

### Example 4: Carbon Dioxide Gas and pH Control

Variations in the carbon dioxide percentage delivered to the culture media to control pH may be used as an indication of metabolic activity of T-cells. The carbon dioxide percentage added to maintain pH (for example, by a controller) can be used to monitor T-cell activation. The carbon dioxide gas percentage, as determined by the pH controller using, for example a proportional-integral control algorithm, may be used to maintain pH at 7.0. Acid side pH control may be accomplished by increasing the carbon dioxide gas concentration in the mixer actuation gas, or generally through sparging gas bubbles in conventional bioreactors, or by delivery of the gas to the headspace of a mixed bioreactor. For TexMACS™ medium, 5% CO₂ is the concentration that results in a media pH value of approximately 7.0. Carbon dioxide gas drive percentage over time is shown in the graph of FIG. 7.

In methods which implement the control of carbon dioxide concentration for pH control, the carbon dioxide drive can be correlated to cell size and the state of cell activation. FIG. 7 shows the carbon dioxide drive for a T-cell culture in the perfusion chamber. Briefly, immediately following cell activation, cellular metabolism increased causing high production of carbon dioxide and acids, reducing the pH and the requirement for supplemental carbon dioxide. As the cells returned to their smaller dormant size, their metabolic activity and acid/carbon dioxide generation rate slowly decreased, as shown by the slow increase in supplemented carbon dioxide required from day 3 to day 8. A second round of activation again showed the behavior of a large increase in metabolic activity and acid/carbon dioxide generation, followed by a slow return to dormant levels of acid/carbon dioxide production.

As shown in FIG. 7, sections of the culture where the carbon dioxide drive was at a minimum correlate with cells acidifying the pH lower than the desired pH setpoint, which is typically pH 7. On day 8 and day 13 of the cultures, the second round of activation resulted in cell mediated media acidification lower than pH 7. In perfusion, a drop in pH below the setpoint can be counteracted by increasing the media flow rate or adding a pH control agent (for example, a base) to increase the pH value. From the data presented in FIG. 7, the percentage of supplemental carbon dioxide may be used to determine the appropriate times for cell activation throughout the expansion process.

While the carbon dioxide drive during activation is generally an indicator of metabolic activity and the degree of cellular activation, there are situations where the carbon dioxide drive is insufficient. To avoid false positives, in embodiments in which the baseline metabolic activity of the cells already drives the pH lower than the setpoint, the base controller drive can be used as an indicator for when cells have finished expanding from the previous activation.

FIG. 7 shows the carbon dioxide demand of the pH controller, where a decrease in carbon dioxide demand is correlated with T-cell activation. The data presented in FIG. 7 shows how online sensor measurements for carbon dioxide drive and pH can be correlated to the metabolic activity of cells, which enables monitoring and control over operations that impact cell metabolism, such as cell activation.

Thus, carbon dioxide drive (optionally, determined by a pH controller), or more generally the pH control drive (acid/base, CO₂/base) may be used as an indicator of metabolic activity, alone or in combination with measurements of optical density as described in Example 3. Metabolic activity can be monitored as an indicator of cell activation and expansion.

### Example 5: Sodium Carbonate pH Control

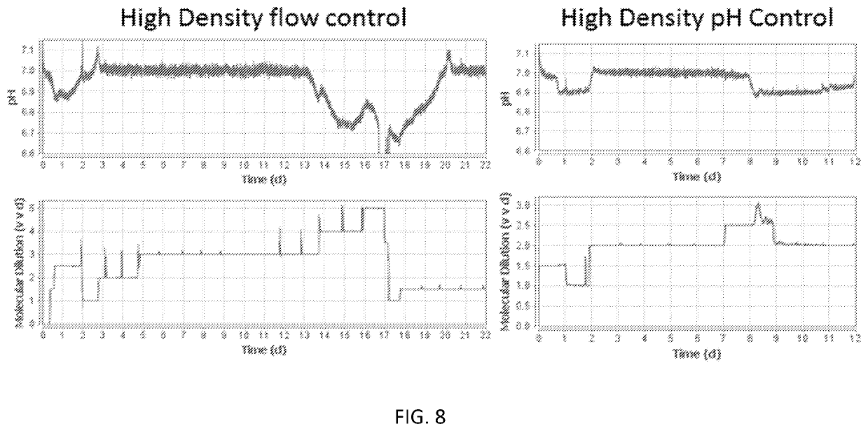

Control of pH during high density T-cell activation was explored. Secondary activation was performed by perfusing TransACT™ into the perfusion chamber having 10M-20M cells/mL. As shown in the data presented in FIG. 8, growth rate decreased responsive to the secondary activation. To control acidification, one cell culture received high flow rate perfusion and another cell culture received sodium carbonate as a pH control agent.

The data on the left of FIG. 8 corresponds to the cell culture receiving high perfusion flow rate to reduce acidification. After the second round of activation (day 13, FIG. 8, left), the cell culture without pH control agent was perfused at flow rates in excess of 5 VVD. The high perfusion cell culture still could not keep up with the cell mediated media acidification. The high perfusion flow rate eventually caused filter clogging and failure of the system to maintain perfusion.

The data on the right of FIG. 8 corresponds to the cell culture receiving sodium carbonate as a pH control agent. After the second round of activation (day 8, FIG. 8, right) the cell culture receiving sodium carbonate as the pH control agent was perfused at flow rates under 3 VVD. The cell culture showed adequate without excessive increase in perfusion flow rate or filter clogging.

Thus, pH control by addition of a pH control agent may mitigate media acidification without substantially increasing perfusion flow rate and/or filter clogging.

### Example 6: Quality and Efficiency of T-Cell Transduction

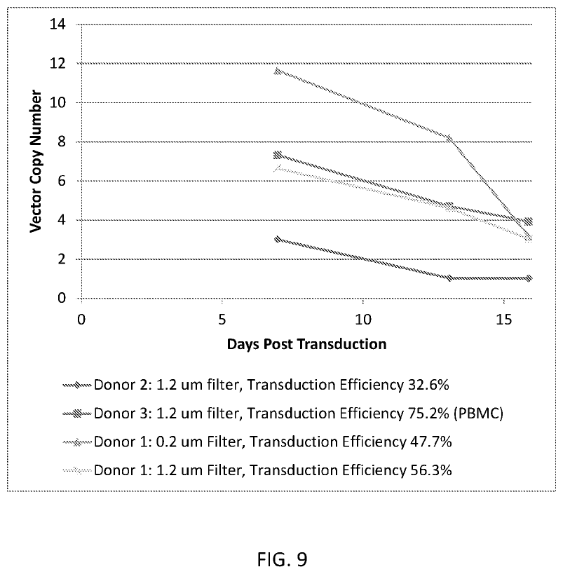

T-cells obtained from three donors were transduced with lentivirus and evaluated. A first sample of transduced T-cells from a first donor were tested with a 0.2 μm filter. A second sample of transduced T-cells from the first donor were tested with a 1.2 μm filter. A third sample of transduced T-cells from a second donor were tested with a 1.2 μm filter. A fourth sample of treated and transduced PBMC from a third donor were tested with a 1.2 μm filter. The results are shown in the graph of FIG. 9. Briefly, the transduction efficiencies were 47.7%, 56.3%, 32.6%, and 75.

FIG. 9 is a graph of the vector copy number (VCN)over time for the four experimental samples described above. Transduction experiments were run using different pore size filters. The 0.2 μm filtered sample VCN started at a much higher post transduction value as compared to the 1.2 μm filtered samples. The results suggest that the filter pore size has an impact on the filterability of the viral particles (here, lentivirus). Even with the virus still present in the perfusion chamber, by day 16, most of the signal from the viral particles was gone. All samples had a lower and more stable VCN.

Thus, filtering the suspension with a filter having a pore size effective to filter the viral particle may reduce and stabilize VCN of the sample.

### Example 7: High Density Perfusion Capability

A perfusion chamber having a 2 mL volume was tested at a high cell density of greater than 40M cells/mL. Conventional T-cell therapy starts from a low-density inoculum, typically ranging from 0.5M to 2M cells/mL or less. With the tested perfusion chamber, harvested T-cells from a patient may be concentrated into the 2 mL working volume and inoculated at high density, rather than at low density. Trial runs of high-density inoculation and transduction were performed and compared to standard low starting cell densities.

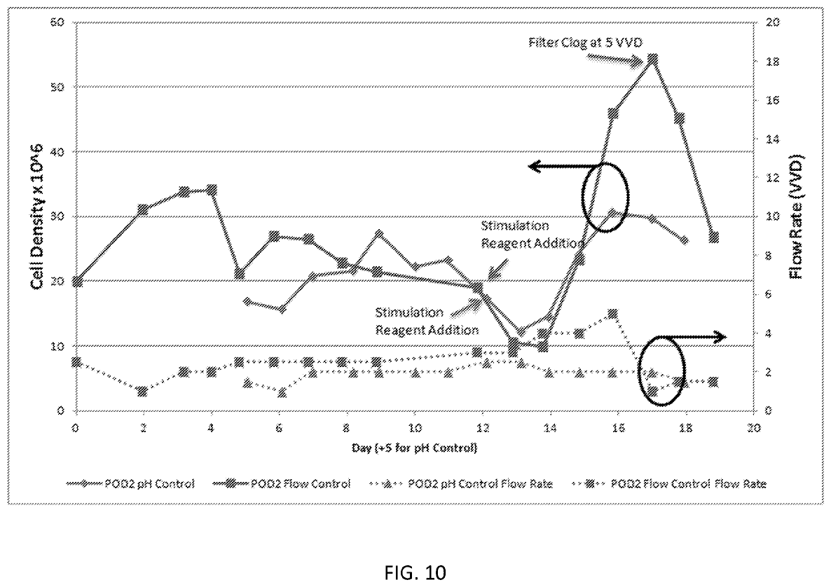

The results of the high-density inoculation are shown in the graph of FIG. 10. Initial activation was performed with a similar protocol to conventional low-density inoculations. The high-density cells were combined with cell activator prior to being introduced into the perfusion chamber. However, by introducing the TransACT™ cell activator into the initial inoculum rather than perfusing through the perfusion chamber, the total delivered TransACT™ to the culture on day 0 was likely not enough to cause significant cell activation and expansion. A second activation with TransACT™ via perfusion of media mixed with TransACT™ was delivered with an amount of cell activator effective to activate cells for further expansion.

Starting the cell expansion process at high cell density could greatly reduce the total time needed for manufacturing a CAR-T based therapy. If the total number of T-cells initially harvested from the donor was on the order of the final dose, transduction could be performed in a highly concentrated inoculum and expansion could be skipped entirely.

### Example 8: Quality and Efficiency of T-Cell Expansion

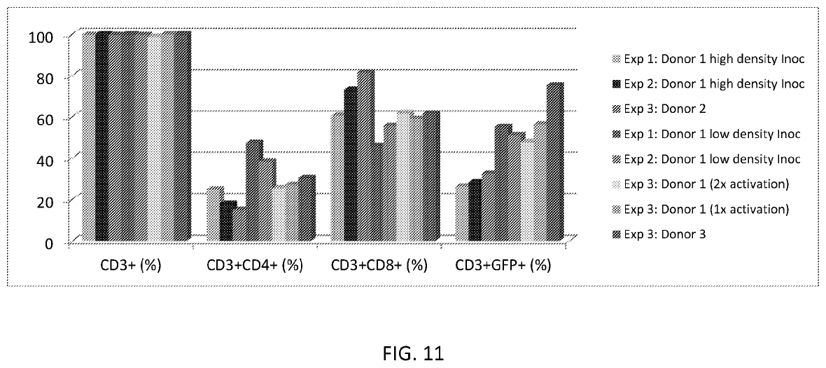

To check the quality of the expansion, the total percentage of CD3+ cells in the perfusion chamber after harvest were assayed to look at the distribution of CD4 and CD8 cells within the CD3 population. The data is shown in the graphs of FIG. 11. Briefly, FIG. 11 includes phenotype data for purity and CD4/CD8 distribution between the samples. Lower GFP transduction efficiency appears to correlate with lower CD4/CD8 ratios.

All samples were transduced with an automated transduction-expansion protocol in the perfusion chamber and were successfully transduced with a GFP producing vector. Two samples were inoculated into the perfusion chambers at a high cell density (20M cells/mL) and infected at a highly reduced multiplicity of infection (MOI). These two samples showed low transduction efficiency.

It was observed that the transduction efficiency in the high-density inoculation samples was lower than the low-density inoculation samples. However, the transduction efficiency in proportion to the MOI was higher in the high-density inoculation samples (4 to 5 active virus particles/cell) than the low-density inoculation samples (53 to 80 active virus particles/cell), indicating that cell density and mixing conditions inside the perfusion chamber likely enhance the transduction efficiency per virus particle in solution.

Thus, transduction efficiency may be enhanced by controlling cell density and mixing conditions within the perfusion chamber.

### Example 9: T-Cell Preparation, Activation, and Transduction Procedure

T-cells were prepared as described in the T-Cell Preparation section below and inoculated into the perfusion chamber at a cell density of 1M cells/mL following the procedure outlined in the Inoculation section below. After one or two days, the cell density was assayed and lentiviral vector with a GFP payload was introduced into the perfusion chamber by first removing a fixed volume of cell-free media from the perfusion chamber (either 500 μL or 1 mL depending on the cell density) and then filling back to a total working volume of 2 mL with a mixture of viral vector either in PBS or PBS supplemented with 5% human serum albumin Daily samples were removed from the perfusion chamber for cell count and viability measurements.

Perfusion of fresh media and removal of waste products started 24 hours after addition of viral vector when the cell density was less than 5M cells/mL. For high cell density inoculation, perfusion was started immediately after inoculation.

An additional cell activation was typically performed on day 11 by switching to culture media containing the cell activation additive.

### Perfusion Chamber Devices

Perfusion chamber devices for the experiment contained a culture chamber comprising three interconnected variable volume sub-chambers, a perfusion filter, optical sensors for pH and dissolved oxygen measurement, and structures to provide low path length optical density measurement. The perfusion chamber further contained a fluid injector section that supported the introduction of four different fluids through four injector input ports, a perfusion outflow section with a suction chamber to suck fluid through the perfusion filter and transport the fluid to a perfusion output port, an output waste port for cell waste, a sample/inoculation input/output port for sampling or manually introducing material, an input port for sterile air purge, and fluid channels connecting the fluid input and output ports to the culture chamber. Pneumatically actuated valves were used to control whether fluid was allowed to flow in the fluid channels.

The variable volume sub-chambers contained a lower chamber and an upper chamber separated by a silicone membrane. The lower chambers were interconnected, allowing fluid communication between the lower chambers. The upper chambers were configured to allow independent pressurization of each upper chamber.

The perfusion chamber devices were fabricated by CNC machining various features such as channels, chambers, and holes, into polycarbonate sheets. The sheets were then bonded together with an intervening silicone membrane approximately 100 μm thick to form fluidic devices such as valves, pumps, and mixing chambers. Additional polycarbonate manifold layers were bonded with adhesive to route the pneumatic signals used to actuate the fluidic devices from the valves and mixing chambers to pneumatic control ports. Completed perfusion chamber devices were sterilized with gamma irradiation.

A controller provided the pneumatic signals to operate the perfusion chamber device and also sent and received optical signals to interrogate the optical sensors of the perfusion chamber device. The controller controlled the temperature of the perfusion chamber device.

The perfusion chamber was configured to perform various operations including: inoculation of cells; culture maintenance with mixing; cell-free liquid exchange to introduce viral vector or activation reagent; addition of fresh nutrients, water, activation reagent, or viral vector through precise fluid injection; cell-free removal of liquid through a cell retention filter; precise control of average perfusion rate through the culture chamber; removal of cell samples, typically less than 5-10% of the working volume; and measurement and control of pH, Dissolved Oxygen, optical density, and temperature.

### Addition of Media Through a Sample/Inoculation Port

Liquid was added to the culture chamber through the sample/inoculation port by first emptying the culture chamber or removing a volume of liquid from the culture chamber, priming the fluid channels between the sample port and culture chamber, then sucking or pumping fluid into the culture chamber. A sample fluid channel connected the sample/inoculation port to a channel junction, a waste fluid channel connected the waste port to the channel junction, and a chamber channel connected the fluid junction to the culture chamber. A sample valve associated with the sample fluid channel, when closed, isolated the sample/inoculation port from the channel junction. A waste valve associated with the waste fluid channel, when closed, isolated the waste port from the channel junction. A chamber valve associated with the chamber channel, when closed, isolated the channel junction and the culture chamber.

Priming the fluid channels was accomplished by connecting a fluid source to the sample/inoculation port, opening the sample and waste valves, then pumping or sucking fluid from the sample/inoculation port to the waste port, then closing the sample and waste valves. To introduce fluid into the culture chamber, the sample valve and chamber valve was opened, and vacuum applied to two of the culture chamber upper chambers to suck fluid from the sample port to the culture chamber.

### Inoculation

A 10 mL syringe was filled with 3 mL of T-cell inoculum prepared as described below. The remaining volume of the syringe was sterile air. The syringe was attached to an inoculation port of the perfusion chamber through a needless valve port. In other embodiments, a luer lock connection or sterile tube welding may also been used. The syringe was positioned such that the liquid inoculum was at the output port of the syringe and the air at the plunger.

The perfusion chamber valves were configured to empty the perfusion chamber by pressurizing the upper chambers of the sub-chambers, then configuring the valves to connect the culture chamber to the waste port. When the sub-chamber membranes were fully deflected into the culture chamber, minimizing the liquid volume of the culture chamber, the perfusion chamber valves were configured to isolate the culture chamber from the input and output ports. The perfusion chamber valves were then configured to connect the sample/inoculation port to the waste port.

The syringe was manually actuated until liquid entered the sample/inoculation port and started to come out of the waste port in order to prime the fluid channels connecting the sample/inoculation port to the culture chamber. The perfusion chamber valves were then configured to connect the sample port and the culture chamber, and vacuum pressure was applied to two of the upper chambers while the third upper chamber remained pressurized. In this configuration, the inoculum was sucked into the culture chamber.

Culture Maintenance with Mixing

Cell cultures were maintained by intermittently mixing the culture chamber. A mixing cycle was accomplished by pressurizing the upper chamber of one of three sub-chambers at a time, changing which upper chamber was pressurized with a frequency between 1.5 Hz and 5 Hz. Typically, 3 to 5 mixing cycles were executed consecutively followed by a delay between 0 seconds and 15 seconds where no mixing occurred.

### Fluid Removal Through Perfusion Filter

A perfusion filter was attached in the culture chamber to prevent particles larger than the perfusion filter pore size to pass between the culture chamber and suction chamber, while allowing liquid and particles smaller than the perfusion filter pore size to pass between the culture chamber and suction chamber. The suction chamber comprised a lower liquid chamber, an upper vacuum chamber, a silicone membrane separating the lower and upper chambers, an inlet, and an outlet. The lower liquid chamber and upper vacuum chamber were arranged such that the outlines of each chamber approximately coincided. By applying pressure or vacuum to the upper chamber, liquid was sucked into or expelled from the suction chamber. Valves at the inlet and the outlet were used to control fluid entry and exit from the inlet and outlet. A fluid removal cycle was performed, including: opening the outlet valve and pressurizing the upper chamber; closing the outlet valve; opening the inlet valve; applying vacuum to the upper chamber; waiting for between 0 and 600 seconds; and closing the inlet valve. The fluid removed per cycle was approximately 10 μL.

### Viral Transduction

To introduce viral vector, 1 mL of culture media was removed through the perfusion filter. The media was removed in cycles, as described above. Briefly, the media was removed by removing fluid through the perfusion filter, and then back filling with a solution containing viral vector. Addition of viral vector was accomplished by filling a syringe with 2 mL of viral vector solution and following the procedure described above with respect to inoculation and introduction of fluids through the sample/inoculation port.

### T-cell Preparation

Peripheral blood mononuclear cells (PBMC) were acquired from apheresis and incubated for 7 days in a G-Rex culture with TexMACS™ media and T-cell TransAct™. The media included 50 U/mL IL-2 to enrich for T-cells. Inoculum was prepared by diluting T-cells to a density of 1M cells/mL in 3 mL of TexMACS™ media, 170 μL of T-cell TransACT™, and 100 U/mL of IL-2. For PBMC inoculation, total cells were diluted to a density of 1M cells/L in 3 mL of TexMACS™ media, 170 μL of T-cell TransACT™, and 100 U/mL of IL-2.

### Lentiviral Vector Preparation

Lentivirus delivering GFP transgene were previously aliquoted and frozen at −80° C. A cell-based assay for infectious particles from thawed aliquots of frozen vector yielded 250M particles/mL.

### Reagents

The T-cell culture medium used was TexMACS™ medium. The cell activator was T-cell TransACT™ polymeric nanomatrix conjugated with humanized CD3 and CD28.

### Analytical Methods

Cell counts and viability were assessed with a NucleoCounter® NC-200™ cell counter (distributed by ChemoMetec, LiHerod, Denmark) using single use Vial-Cassettes.

Transduction efficiency was assayed by flow cytometry to count the fraction of GFP expressing cells.

Average vector copy number (VCN) per cell in the population was assayed using a qPCR technique. Briefly, the quantity of vector gene was compared to the quantity of human albumin gene. The quantity of vector gene and human albumin gene was determined by comparison to standard curves generated by serial dilution of plasmids with known copy number. The assay was performed on cell samples including transduced and untransduced cells.

Cell surface marker phenotypes were assayed by flow cytometry utilizing labels for CD3, CD4, and CD8.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure, and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. A method of treating cells, comprising:
introducing a media comprising at least about 3×10⁶ cells/mL into a perfusion chamber having a volume of 50 mL or less;
perfusing the cells by:
introducing a volume effective to treat the cells of at least one additive selected from cell culture media, a transducing agent, a pH control agent, and a cell activator into the perfusion chamber; and
withdrawing cell waste and byproducts from the perfusion chamber; and harvesting the treated cells.

2. The method of claim 1, wherein the media comprises between about 5×10⁶ cells/mL and about 20×10⁶ cells/mL.

3. The method of claim 2, wherein the perfusion chamber has a volume of 20 mL or less.

4. The method of claim 3, wherein the perfusion chamber has a volume of 2.5 mL or less.

5. The method of claim 1, wherein the additive comprises the pH control agent, and the method further comprises controlling pH of the media within the perfusion chamber to a pH value of between about 6.8 and 7.4.

6. The method of claim 1, wherein the at least one additive is introduced at a flow rate of 5 volumes of fluid per volume of reactor per day (VVD) or less.

7. The method of claim 6, wherein the at least one additive is introduced at a flow rate of between about 1 VVD and about 3 VVD.

8. The method of claim 1, further comprising introducing additional cells into the perfusion chamber and concentrating the cells within the perfusion chamber.

9. The method of claim 8, comprising concentrating the cells to a concentration of at least about 5×10⁶ cells/mL.

10. The method of claim 9, comprising concentrating the cells to a concentration of at least about 10×10⁶ cells/mL.

11. The method of claim 10, comprising concentrating the cells to a concentration of at least about 20×10⁶ cells/mL.

12. The method of claim 1, wherein the harvested treated cells have a viability of at least about 60%.

13. The method of claim 12, wherein the harvested treated cells have a viability of at least about 90%.

14. The method of claim 12, wherein at least about 60% of the harvested cells are effectively treated.

15. The method of claim 14, wherein at least about 90% of the harvested cells are effectively treated.

16. A method of treating cells, comprising:
introducing a media comprising at least about 0.5×10⁶ cells/mL into a perfusion chamber having a volume of 50 mL or less;
measuring at least one parameter of the cells or the media, the at least one parameter selected from pH, optical density, dissolved oxygen concentration, temperature, and light scattering;
determining a cell state associated with at least one of metabolic activity of the cells, average size of the cells, and density of the cells in the media, responsive to the measurement of the at least one parameter;
introducing a volume effective to treat the cells of at least one additive selected from cell culture media, a transducing agent, a pH control agent, and a cell activator into the perfusion chamber, the volume effective of the at least one additive selected responsive to the cell state; and harvesting the treated cells.

17. The method of claim 16, wherein the media comprises at least about 3×10⁶ cells/mL.

18. The method of claim 16, wherein the perfusion chamber has a volume of 2.5 mL or less.

19. The method of claim 16, wherein the method comprises measuring the pH and introducing a volume effective of a pH control agent to control the pH to be between about 6.8 and 7.4.

20. The method of claim 19, wherein the method comprises quantifying a volume of carbon dioxide gas introduced into the perfusion chamber to control the pH to be between about 6.8 and 7.4.

21. The method of claim 16, wherein the additive comprises the transducing agent and the method further comprises introducing an effective volume of a transduction efficiency enhancing agent.

22. The method of claim 16, comprising determining the cell state associated with metabolic activity of the cells responsive to the measurement of the at least one parameter selected from pH and optical density; and
introducing the volume effective of the at least one additive selected from the transducing agent and the cell activator into the perfusion chamber, responsive to the cell state.

23. The method of claim 16, comprising determining the cell state associated with the density of the cells in the media responsive to the measurement of the at least one parameter selected from optical density and light scattering.

24. A method of treating cells, comprising:
introducing a media comprising at least about 0.5×10⁶ cells/mL into a perfusion chamber having a volume of 50 mL or less;
perfusing the cells by:
introducing a first volume of at least one additive selected from cell culture media, a transducing agent, a pH control agent, and a cell activator into the perfusion chamber;
after a first predetermined period of time, introducing a second volume of the at least one additive; and
after a second predetermined period of time, withdrawing cell waste and byproducts from the perfusion chamber; and
harvesting the treated cells.

25. The method of claim 24, wherein the media comprises at least about 3×10⁶ cells/mL.

26. The method of claim 24, wherein the perfusion chamber has a volume of 2.5 mL or less.

27. The method of claim 24, wherein at least one of the first and second predetermined period of time is less than about 1 hour.

28. The method of claim 27, wherein the first predetermined period of time is less than about 1 minute.

29. The method of claim 28, wherein the first predetermined period of time is less than about 15 seconds.
